(19) United States
(12) Patent Application Publication
(10) Pub. No.: US 2016/0038760 A1
(43) Pub. Date: Feb. 11, 2016

(54) CONJUNCTIVAL DISEASES

(71) Applicant: MASSACHUSETTS EYE AND EAR INFIRMARY, Boston, MA (US)

(72) Inventors: Pedram Hamrah, Wellesley, MA (US); Ahmad Kheirkhah, Boston, MA (US)

(21) Appl. No.: 14/774,176

(22) PCT Filed: Mar. 5, 2014
(86) PCT No.: PCT/US14/20594

§ 371 Date: Sep. 10, 2015

Related U.S. Application Data
(60) Provisional application No. 61/783,659, filed on Mar. 14, 2013.

(51) Int. Cl. A61N 5/06; A61F 9/008

(52) U.S. Cl. CPC A61N 5/062; A61F 9/008; A61N 2005/0661; A61N 2005/067

(57) ABSTRACT

Provided herein are methods of treating conjunctival diseases, and methods of increasing the rigidity of a conjunctiva, decreasing the proliferation or number of conjunctival fibroblasts in a conjunctiva, and decreasing the number of immune cells present in a conjunctiva in a subject.

### CROSS-REFERENCE TO RELATED APPLICATIONS

This application claims priority to U.S. Provisional Patent Application Ser. No. 61/783,659, filed Mar. 14, 2013, the entire contents are herein incorporated by reference.

### BACKGROUND OF THE INVENTION

The conjunctiva is a tissue that lines the insides of the eyelids and covers the sclera, and is composed of non-keratinized, stratified columnar epithelial cells. The conjunctiva allows free movement of the globe of the eye.

A variety of conjunctival diseases affect humans. These include, for example, different inflammatory, proliferative, or degenerative diseases. The most common degenerative disease is conjunctivochalasis (CCH) or conjunctival wrinkles, which is characterized by a loose redundant bulbar conjunctiva that interposes between the lids and globe of the eye, more commonly in the inferior conjunctiva. A community-based study showed that 44% of people 60 years or older have CCH (Zhang et al., BMC Public Health 11:198, 2011), and another hospital-based study showed that 98.5% of people 60 years of older have CCH (Mimura et al., Am. J. Ophthalmol. 147(1):171-177, 2009). CCH is one of the most common causes of ocular surface irritation with a wide variety of symptoms including the feeling of dryness, tearing, foreign body sensation, and burning. Although CCH symptoms may be controlled by topical medications such as steroids and lubrication, for refractory cases surgical procedures are currently performed in order to relieve the symptoms of subjects having CCH.

### SUMMARY OF THE INVENTION

The invention is based, at least in part, on the concept that topically administering (or subconjunctivally injecting) a crosslinking agent to an eye of a subject, and irradiating a subsection of the conjunctiva in the eye of the subject with ultraviolet A (UVA) light can result in one or more of an increase in the rigidity of the conjunctiva, a decrease in the proliferation or number of conjunctival fibroblasts in a conjunctiva, and/or a decrease in the number of immune cells present in a conjunctiva, and therefore such administering and irradiating can successfully treat a variety of different conjunctival diseases.

In view of these discoveries, provided herein are methods of treating a conjunctival disease, increasing the rigidity of a conjunctiva, decreasing the proliferation or number of conjunctival fibroblasts in a conjunctiva, and decreasing the number of immune cells present in a conjunctiva that include topically administering (or subconjunctivally injecting) a composition containing a crosslinking agent to an eye of a subject (e.g., a subject having or suspected of having a conjunctival disorder), and irradiating a subsection of the conjunctiva in the eye of the subject with UVA light.

Provided herein are methods of treating a conjunctival disease in a subject that include topically administering or subconjunctivally injecting a composition containing a crosslinking agent into an eye of a subject having or suspected of having a conjunctival disease; and irradiating a subsection of the conjunctiva of the eye of the subject with ultraviolet A (UVA) light for a time and duration sufficient to treat the conjunctival disease in the eye of the subject. In some embodiments of any of the methods described herein, the conjunctival disease is selected from the group of: conjunctivochalasis, conjunctival bleb thinning a glaucoma surgical procedure, pterygium, a cicatricial conjunctival disorder, autoimmune disease of conjunctiva, allergic conjunctivitis, and dry eye disease.

Also provided are methods of increasing the rigidity of a conjunctiva in a subject that include topically administering or subconjunctivally injecting a composition containing a crosslinking agent into an eye of a subject having or suspected of having decreased rigidity in conjunctiva of the eye of the subject; and irradiating a subsection of the conjunctiva of the eye of the subject with ultraviolet A (UVA) light for a time and duration sufficient to increase the rigidity of the conjunctiva in the eye of the subject.

Also provided are methods of decreasing the proliferation or number of conjunctival fibroblasts in a conjunctiva in a subject that include topically administering or subconjunctivally injecting a composition containing a crosslinking agent into an eye of a subject having or suspected of having increased proliferation or number of conjunctival fibroblasts in conjunctiva of the eye of the subject; and irradiating a subsection of the conjunctiva of the eye of the subject with ultraviolet A (UVA) light for a time and duration sufficient to decrease the proliferation or number of conjunctival fibroblasts in the subsection of the conjunctiva of the eye of the subject.

Also provided are methods decreasing the number of immune cells in a conjunctiva in a subject that include topically administering or subconjunctivally injecting a composition containing a crosslinking agent into an eye of a subject having or suspected of having an increased number of immune cells in conjunctiva of the eye of the subject; and irradiating a subsection of the conjunctiva of the eye of the subject with ultraviolet A (UVA) light for a time and duration sufficient to decrease the number of immune cells in the subsection of the conjunctiva of the eye of the subject.

In some embodiments of any of the methods described herein, the irradiating is performed about 1 second to about 2 hours after the administering. In some embodiments of any of the methods described herein, the administering is performed intermittently during the irradiating period. In some embodiments of any of the methods described herein, the administering is performed both before and intermittently during the irradiating period.

In some embodiments of any of the methods described herein, the crosslinking agent is riboflavin, rose bengal, octadiazopyruvoyl PAMAM (8G.1 DAP, 1.3), or a 1,8-naphthalimide dye. In some embodiments of any of the methods described herein, the irradiating is performed using a laser that emits UVA light. In some embodiments of any of the methods described herein, the irradiating is performed using a double diode laser. In some embodiments of any of the methods described herein, the UVA light only irradiates the conjunctiva to a maximum depth of 250 μm from the surface of the conjunctiva. In some embodiments of any of the methods described herein, the UVA light only irradiates the conjunctiva to a maximum depth of 200 μm from the surface of the conjunctiva. In some embodiments of any of the methods described herein, the UVA light does not irradiate the sclera of the eye of the subject.

Some embodiments of any of the methods described herein further include positioning a mask or filter over a portion of the eye in order to prevent the UVA light from irradiating the sclera, cornea, and/or limbus. In some embodiments of any of the methods described herein, the subsection of the conjunctiva is irradiated for less than about 1 hour. In some embodiments of any of the methods described herein, the conjunctiva is irradiated for less than 10 minutes.

In some embodiments of any of the methods described herein, the irradiance of the UVA light is between about 0.1 mW/cm² and about 45 mW/cm². In some embodiments of any of the methods described herein, the irradiance of the UVA light is between about 0.1 mW/cm² and about 15 mW/cm². In some embodiments of any of the methods described herein, the UVA light is pulsed UVA light.

In some embodiments of any of the methods described herein, the irradiating provides a dose of between about 0.1 J/cm² to about 15 J/cm². In some embodiments of any of the methods described herein, the irradiating provides a dose of between about 0.1 J/cm² and about 8 J/cm². In some embodiments of any of the methods described herein, the UVA light has a wavelength of about 340 nm to about 380 nm. In some embodiments of any of the methods described herein, the subsection of the conjunctiva has an area of between about 3 mm² to about 300 mm².

In some embodiments of any of the methods described herein, the subject has or is suspected of having conjunctivochalasis or conjunctival bleb thinning after a glaucoma surgical procedure. In some embodiments of any of the methods described herein, the subject has or is suspected of having pterygium or a cicatricial conjunctival disorder. In some embodiments of any of the methods described herein, the cicatricial conjunctival disorder is selected from the group consisting of: Stevens-Johnson syndrome, ocular cicatricial pemphigoid, and thermal or chemical burn of the conjunctiva. In some embodiments of any of the methods described herein, the subject has or is suspected of having an autoimmune disease of conjunctiva, allergic conjunctivitis, and dry eye disease.

Some embodiments of any of the methods described herein further include administering to the subject at least one additional agent for treating a conjunctival disease in the subject. In some of the embodiments described herein, the combination of the administering and irradiating are performed more than twice on the same subject. In some embodiments of any of the methods described herein, the combination of the administering and irradiating are performed at periodic intervals on the same subject.

In some embodiments of any of the methods described herein, the composition containing a crosslinking agent is a hypotonic solution. Some embodiments of any of the methods described herein further include topically administering or subconjunctivally injecting to the eye of the subject a hypotonic solution prior to irradiating the subsection of the conjunctiva in the eye of the subject. Some embodiments of any of the methods described herein further include topically administering or subconjunctivally injecting to the eye of the subject a hypotonic solution both before and intermittently during the irradiating of the subsection of the conjunctiva in the eye of the subject. Some embodiments of any of the methods described herein further include topically administering or subconjunctivally injecting to the eye of the subject a hypotonic solution during the irradiating of the subsection of the conjunctiva in the eye of the subject.

By the term “ultraviolet A light” or “UVA light” is meant light having only one or more specific wavelengths that fall between 315 nm and 400 nm (inclusive). Non-limiting sources of UVA light and exemplary parameters of UVA light exposure are described herein. Additional sources of UVA light and exemplary parameters of UVA light exposure are known in the art.

By the term “conjunctival disease” is meant an abnormality in conjunctival tissue or function in a subject that results in or is caused by a decrease in the rigidity of a conjunctiva as compared to the rigidity of a conjunctiva of a healthy subject, increased proliferation or number of conjunctival fibroblasts as compared to proliferation and number of conjunctival fibroblasts in a healthy subject, and/or an elevated number of immune cells in a conjunctiva as compared to the number of immune cells in a conjunctiva of a healthy subject. Non-limiting examples of conjunctival diseases include conjunctivochalasis, allergic conjunctivitis, pterygium, ocular cicatricial pemphigoid, and other inflammatory, but non-infectious conjunctival diseases. Conjunctival diseases are also typically characterized by a decrease (e.g., an observable, detectable, or significant decrease) in conjunctival secretion of mucus and/or tears that lubricate the eye. Non-limiting examples of conjunctival diseases are described herein. Additional examples of conjunctival diseases are known in the art.

By the term “immune cells” is meant any type of immune cell that can infiltrate a conjunctiva. Non-limiting examples of immune cells include dendritic (Langerhans) cells, eosinophils, mast cells, and T- and B-lymphocytes.

By the term “allergic conjunctivitis” is meant a set of hypersensitivity disorders of the immune system that are triggered by an allergen and are characterized by at least one of the symptoms selected from the group of swelling, redness, itching, and irritation of the conjunctiva. Several of the symptoms of allergic conjunctivitis are mediated by the activation of mast cells and basophils in a subject (e.g., in a conjunctiva of the subject) that release several mediators into the tissue of the subject (e.g., one or more mediators selected from the group consisting of: serine proteases, histamine, serotonin, heparin, thromboxane, prostaglandin D2, leukotriene C4, platelet-activating factor, and eosinophil-activating factor). Non-limiting examples of allergic conjunctivitis are atopic keratoconjunctivitis, vernal keratoconjunctivitis, seasonal allergic conjunctivitis, perennial allergic conjunctivitis, and giant papillary conjunctivitis (GPC).

By the term “autoimmune disease of conjunctiva” is meant a disease that is caused by an improper immune response in a subject that targets his or her conjunctiva.

By the term “atopic keratoconjunctivitis” or “AKC” is meant a form of conjunctivitis caused by allergy. In some embodiments, AKC is a bilateral chronic disease that is associated with atopic dermatitis. Atopic keratoconjunctivitis is a non-limiting example of an allergic conjunctivitis.

By the term “vernal keratoconjunctivitis” or “VKC” is meant a severe form of conjunctivitis caused by allergy. In some embodiments, VKC is a bilateral chronic disease of conjunctiva (e.g., without skin involvement). Vernal keratoconjunctivitis is a non-limiting example of an allergic conjunctivitis.

By the term “subject” is meant any mammal (e.g., a human, mice, rat, or rabbit).

By the term “healthy subject” is meant a mammal (e.g., a human) that does not have an eye, eyelid, corneal, or conjunctival disease.

By the term “crosslinking agent” is meant a molecule that absorbs ultraviolet A (UVA) light and is capable of crosslinking collagen protein. Non-limiting crosslinking agents are described herein. Additional examples of crosslinking agents are known in the art.

By the term “cicatricial conjunctival disorder” is meant a conjunctival disease that results in conjunctival scarring, and in some instances can, e.g., cause symblepharon. Non-limiting examples of cicatricial conjunctival disorders include Stevens-Johnson syndrome, ocular cicatricial pemphigoid, and thermal or chemical burns in the conjunctiva.

By the term “pulsed UVA light” or “pulsed ultraviolet A light” is meant a periodic irradiation of UVA light, where the irradiation of UVA light is performed and ceased at intervals (e.g., a fixed frequency). Non-limiting examples of pulsed UVA light are described herein.

By the term “rigidity of conjunctiva” is an art-recognized term and means the level of adhesion between the conjunctiva and underlying sclera. The rigidity of a conjunctiva can be assessed, e.g., by clinical examination and/or biomechanical evaluation (e.g., using methods known by those in the art). Additional non-limiting methods for assessing rigidity of the conjunctiva in a subject are described herein (e.g., in the Examples). A subject having a reduced rigidity of a conjunctiva may present with eye irritation and/or has a disruption in his or her tear film.

Other definitions appear in context throughout this disclosure. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION OF THE INVENTION

The invention is based on the concept that topically administering (or subconjunctivally injecting) a crosslinking agent to an eye of a subject, and irradiating a subsection of the conjunctiva in the eye of the subject with ultraviolet A (UVA) light can result in one or more of an increase in the rigidity of the conjunctiva, a decrease in the proliferation or number of conjunctival fibroblasts in a conjunctiva, and/or a decrease in the number of immune cells present in a conjunctiva, and therefore such administering and irradiating can successfully treat a variety of different conjunctival diseases. Thus, provided herein are methods of treating a conjunctival disease, increasing the rigidity of a conjunctiva, decreasing the proliferation or number of conjunctival fibroblasts in a conjunctiva, and decreasing the number of immune cells present in a conjunctiva. These methods include topically administering (or subconjunctivally injecting) a composition containing a crosslinking agent to an eye of a subject (e.g., a subject having or suspected of having a conjunctival disorder), and irradiating a subsection of the conjunctiva in the eye of the subject with UVA light. Non-limiting aspects and embodiments of these methods are described herein. Any of the aspects described below can be used in any combination in the methods described herein.

### Conjunctival Diseases

Conjunctival diseases are characterized by an abnormality in conjunctival tissue or function in a subject that results in or is caused by a decrease in the rigidity of a conjunctiva as compared to the rigidity of a conjunctiva of a healthy subject, increased proliferation or number of conjunctival fibroblasts as compared to the proliferation and number of conjunctival fibroblasts in a healthy subject, and/or an elevated number of immune cells in a conjunctiva as compared to the number of immune cells in a conjunctiva of a healthy subject. Conjunctival diseases are also often characterized by a decrease (e.g., an observable, detectable, or significant decrease) in conjunctival secretion of mucus and/or tears that lubricate the eye. Non-limiting symptoms of various conjunctival diseases are described below.

A conjunctival disease can be diagnosed in a subject by assessing one or more (e.g., two, three, or four) symptoms of a conjunctival disorder (e.g., any one or more of the exemplary symptoms listed below for one or more of the exemplary conjunctival diseases). In addition to the observation of one or more of the exemplary symptoms described herein, a subject can be diagnosed as having a conjunctival disease by detecting the conjunctival folds (e.g., in a subject having CCH) and chronic conjunctival inflammation through clinical examination, and/or by identifying ocular surface damage (e.g., using the corneal fluorescein staining, tear break-up time, conjunctival staining by lissamine green and rose bengal, and symptom questionnaires such as Ocular Surface Disease Index (OSDI) and Dry Eye Questionnaire (DEQ) methods known in the art).

In some embodiments, a subject can be diagnosed as having a conjunctival disease by a health care professional (e.g., a physician, a physician's assistant, a nurse, a nurse's assistant, and a laboratory technician). In some embodiments, a subject diagnosed as having a conjunctival disease can be a child, a teenager, or an adult (e.g., at least 18, 20, 25, 30, 35, 40, 45, 50, 55, 60, 65, 70, 75, 80, 85, or 90 years old). A subject diagnosed as having a conjunctival disease may present with one or more (e.g., at least two, three, or four) of any combination of the symptoms of a conjunctival disease described herein. In some embodiments, a subject having a conjunctival disease may not present with a symptom of a conjunctival disorder that can be easily detected by basic examination of an eye(s) (e.g., a conjunctiva) of the subject (examination of the patient that does not involve the magnification of the tissues of the eye).

In some embodiments, the subject has previously received a treatment for a conjunctival disease. In some embodiments, the subject has previously responded poorly to one or more other treatments for a conjunctival disease. A description of some non-limiting examples of conjunctival disorders is provided below.

### Conjunctivochalasis

Conjunctivochalasis (CCH) is characterized in part by a decrease in the rigidity of a conjunctiva as compared to the rigidity of a conjunctiva of a healthy subject (e.g., a subject that does not have an eye or conjunctival disorder). Conjunctivochalasis is typically further characterized by a loose redundant bulbar conjunctiva that interposes between the eyelid and the globe of the eye, more commonly in inferior conjunctiva. Conjunctivochalasis results in ocular surface irritation.

Conjunctivochalasis can be caused by both a gradual loosening of the conjunctiva that accompanies age and loss of adhesion between the conjunctiva and underlying sclera related from the dissolution of the Tenon's capsule. The resulting conjunctiva with reduced rigidity can mechanically irritate the eye and disrupt the tear film and its outflow, leading to both dry eye and excess tearing.

Conjunctivochalasis can be diagnosed in a subject by assessing one or more (e.g., two, three, or four) symptoms selected from the group of: ocular surface irritation, feeling of dryness in the eye, tearing, foreign body sensation, redness, localized pain, subconjunctival hemorrhage, ulceration, irregularities in the tear film, and burning. Diagnosis of conjunctivochalasis can also be made by observation of redundant conjunctival folds during slit lamp examination (e.g., slit lamp examination using fluorescein dye or lissamine green dye). Diagnosis of conjunctivochalasis can also be made by using fluorescein dye to enhance the visibility of the conjunctival folds by showing the irregularities in the tear film. Currently, there is no treatment to halt the progression of conjunctival looseness associated with CCH which occurs over time. Cases with CCH whose symptoms cannot be relieved by medical treatments, such topical anti-inflammatory medications or lubrications, are typically treated by surgical procedures to remove the loose conjunctiva and/or increase the rigidity of the conjunctiva in the eye of the subject. In some embodiments of any of the methods described herein, the subject has been diagnosed as having conjunctivochalasis. In some embodiments of any of the methods described herein, the subject has or is suspected of having conjunctivochalasis.

Methods of treating conjunctival disorders, including conjunctivochalasis, are described herein. Any of the methods described herein can result in an increase in the rigidity of the conjunctiva in the eye of a subject having conjunctivochalasis.

Conjunctival Bleb Thinning after a Glaucoma Surgical Procedure

Conjunctival thinning after a glaucoma surgical procedure (e.g., trabeculectomy) is characterized in part by a decrease in the rigidity of the conjunctiva as compared to a healthy subject (e.g., a subject that does not have an eye or conjunctival disease). For example, after filtering surgery is performed on subjects having glaucoma (e.g., trabeculectomy), a conjunctival bleb is produced to help reduce the intraocular pressure. During these surgical procedures, antimetabolites (e.g., mitomycin C) may be administered to the subjects. Using antimetabolites during these surgeries may result in significant thinning of the walls of the conjunctival blebs. The thinning of the walls of the conjunctival blebs can lead to the formation of microperforations in the conjunctival blebs, which can in turn, result in significant complications such as low intraocular pressure and infection in the bleb or in the eye itself. In some embodiments, the conjunctival bleb thinning can, e.g., result in a decrease in the rigidity of the conjunctiva in the eye of the subject.

Conjunctival bleb thinning after a glaucoma surgical procedure (e.g., trabeculectomy) can be diagnosed in a subject by assessing one or more (e.g., two, three, or four) symptoms selected from the group of: ocular surface irritation, redness, localized inflammation, reduced vision, and even significant infection of the eye structures. In some embodiments of any of the methods described herein, the subject has been diagnosed as having conjunctival bleb thinning after a glaucoma surgical procedure (e.g., trabeculectomy). In some embodiments of any of the methods described herein, the subject has or is suspected of having conjunctival bleb thinning after a glaucoma surgical procedure (e.g., trabeculectomy). In some embodiments of any of the methods described herein, a glaucoma surgical procedure (e.g., trabeculectomy) was performed on the subject in the last year (e.g., in the last 10 months, in the last 8 months, in the last 6 months, in the last 4 months, in the last two months, in the last month, or in the last two weeks). Currently, there is no treatment to halt the progression of conjunctival bleb thinning after a glaucoma surgical procedure (e.g., trabeculectomy).

The methods described can be used to treat a conjunctival disease, including a conjunctival bleb thinning after a glaucoma surgical procedure (e.g., trabeculectomy). The methods described herein can result in an increase in the rigidity of a conjunctiva (e.g., as compared to the rigidity in the subject prior to treatment) in a subject having conjunctival bleb thinning after a glaucoma surgical procedure (e.g., trabeculectomy). In addition, the methods described herein can also decrease the conjunctival/Tenon fibroblast number and/or proliferation at the filtering site (in the eye of the subject) in a trabeculectomy procedure and thereby improve the outcome of surgery. Any of the methods described herein provide for an improvement in the clinical outcome of glaucoma surgery in a subject (e.g., one or more of decrease in conjunctival bleb thinning, an increase in the rigidity of the conjunctiva, a decrease in the formation of microperforations in the conjunctival blebs, and a decrease the conjunctival/Tenon fibroblast number and/or proliferation (e.g., scar formation) at the surgical site (e.g., the filtering site in the eye of the subject having had a trabeculectomy).

### Pterygium

Pterygium is caused by an abnormal fibrovascular proliferation (e.g., a proliferation of conjunctival fibroblasts) that extends from the conjunctiva onto the cornea of an eye of a subject. Pterygium can be diagnosed in a subject by assessing one or more (e.g., two, three, or four) symptoms selected from the group of: presence of a fibrovascular tissue formed or forming over the cornea, ocular irritation, disfigurement, vision loss, increase in astigmatism, and scarring. Currently, there is no treatment to cause regression of the fibrovascular proliferation in pterygium. Pterygium is typically treated by surgical resection of the fibrovascular tissue formed over the cornea and treatment with grafts and/or antimetabolite agents (e.g., mitomycin C). Treatment of pterygium with antimetabolite agents can result in significant long-term complications, such as scleral melting and ischemia.

In some embodiments of the methods described herein, a subject that is treated can have primary or recurrent pterygium. In some embodiments of any of the methods described herein, the subject has been diagnosed as having pterygium. In some embodiments, the subject has or is suspected of having pterygium. In some embodiments of the methods described herein, the subject has an early stage of pterygium or has previously had (e.g., within the last six months) surgery to resect fibrovascular tissue from the surface of the cornea. In some embodiments, the subject has previously received a treatment for pterygium, and the subject was not responsive or had a poor response to the treatment.

Any of the methods described herein can be used to treat a conjunctival disease, including pterygium in a subject. The methods described herein can induce apoptosis of fibroblasts in the conjunctiva and/or reduce the proliferation of fibroblasts in the conjunctiva (e.g., fibroblasts in the fibrovascular tissue formed over the cornea in a subject having pterygium), and thereby result in a decrease in the number and/or proliferation of conjunctival fibroblasts (e.g., decrease the number and/or proliferation of conjunctival fibroblasts in a subject having pterygium). The methods described herein can result in regression of the fibrovascular tissue in pterygium without surgical resection, reduce fibrovascular proliferation after the resection (thereby reducing the recurrence rate of pterygium after the resection), and/or reduce fibrovascular proliferations in the early forms of the recurrence of pterygium after the resection.

### Cicatricial Conjunctival Disorders

Cicatricial conjunctival disorders are conjunctival diseases that result in conjunctival scarring, and in some instances can, e.g., cause symblepharon. The scar tissue in the conjunctiva of the cicatricial conjunctival disorders is formed following, in part, an increased proliferation of fibroblasts in the conjunctiva, which results in both an increased number of fibroblasts and scar tissue in the conjunctiva. As is known in the art, a symblepharon is at least a partial adhesion of the palpebral conjunctiva of the eyelid to the bulbar conjunctiva of the eyeball that is caused, in part, by the increased proliferation of fibroblasts in the conjunctiva. Non-limiting examples of cicatricial conjunctival disorders include without limitation: Stevens-Johnson syndrome, ocular cicatricial pemphigoid, thermal burns in a conjunctiva, and chemical burns in a conjunctiva.

Cicatricial conjunctival disorders can be diagnosed in a subject by assessing one or more (e.g., two, three, or four) symptoms selected from the group of: burning eyes, conjunctival inflammation, conjunctival scarring, peripheral corneal thinning, dry eye, impaired vision, gritty eye sensation, eye pain, scarred cornea, red eyes, tearing, light-sensitive eyes, adhesion of eyelid to eye, corneal ulceration, corneal perforation, conjunctival ischemia, conjunctival irritation, limitation of eye motility, and blurred vision. Currently, there is no treatment to prevent progression of scar formation in cicatricial conjunctival disorders except for systemic immunosuppressive treatment for cases with systemic autoimmune disease. For cases with established scarring and, e.g., symblepharon, the only treatment modality is surgery to remove the scar tissue, which has a high risk of recurrence of scar tissue. To prevent recurrence of conjunctival scarring, antimetabolite agents (such as mitomycin C) may be used. However, these agents may be associated with significant long-term complications.

In some embodiments of the methods described herein, the subject has an early stage of a cicatricial conjunctival disorder. In some embodiments, the subject was exposed (e.g., within the last week, e.g., within 2 days or within 1 day) to an agent that would induce a chemical burn in a conjunctiva. In some embodiments, the subject has been exposed (e.g., within the last week, e.g., within 2 days or within 1 day) to heat source or temperature that would result in a thermal burn in a conjunctiva. In some embodiments, the subject has been diagnosed with a cicatricial conjunctival disorder (e.g., any of the cicatricial conjunctival disorders described herein or known in the art). In some embodiments, the subject has or is suspected of having a cicatricial conjunctival disorder (e.g., any of the cicatricial conjunctival disorders described herein or known in the art).

Provided herein are methods of treating cicatricial conjunctival disorders, including any of the cicatricial conjunctival disorders described herein or known in the art. The methods provided herein can decrease the proliferation or number of conjunctival fibroblasts (e.g., the fibroblasts that form scar tissue in a conjunctiva or the symblepharon) in subjects having any of the cicatricial conjunctival disorders described herein or known in the art. By reducing the number and proliferation of fibroblasts, the methods described herein can halt the progression of the cicatricial conjunctival disorders. The methods described herein can also help reduce fibrovascular proliferation after the resection of scar tissue and thereby reducing the recurrence rate of scarring after the resection surgery. The methods described herein can also reduce fibrovascular proliferations in the early forms of the recurrence of scarring soon after the resection surgery.

### Autoimmune Disease of Conjunctiva

Autoimmune disease of conjunctiva is caused by an improper immune response in a subject that targets his or her conjunctiva. An autoimmune disease of conjunctiva can be characterized by inflammation or infiltration of one or more types of immune cells (e.g., macrophages, dendritic (Langerhans) cells, mast cells, neutrophils, and T- and B-lymphocytes) to the conjunctiva. An autoimmune disease of conjunctiva can also be associated with increased levels of one or more (e.g., two, three, four, or five) different pro-inflammatory cytokines (e.g., IL-1 and TNFα) in the conjunctiva, tears, and/or in a tissue proximal to the conjunctiva.

Autoimmune disease of conjunctiva can be diagnosed in a subject by assessing one or more (e.g., two, three, or four) symptoms selected from the group of: eye pain, conjunctival swelling, impairment of sight, conjunctival inflammation, dry eye, and red eye. In some embodiments of the methods described herein, the subject can have an early stage of an autoimmune disease of conjunctiva. In some embodiments, the subject has been diagnosed as having an autoimmune disease of conjunctiva. In some embodiments, the subject has or is suspected of having an autoimmune disease of conjunctiva. In some embodiments, the subject has previously been administered a treatment for an autoimmune disease of conjunctiva. In some embodiments, the subject has previously been administered a treatment for an autoimmune disease of conjunctiva and the subject was not responsive or had a poor response to the treatment.

Any of the methods described herein can be used to treat a conjunctival disease, such as an autoimmune disease of conjunctiva. Any of the methods described herein can result in a decrease in the number of immune cells (e.g., any of the exemplary immune cells described herein) in a conjunctiva in a subject having an autoimmune disease of conjunctiva and/or can result in a decrease in the migration of immune cells (e.g., any of the exemplary immune cells described herein) in a conjunctiva in a subject having an autoimmune disease of conjunctiva.

### Allergic Conjunctivitis

Allergic conjunctivitis is a set of hypersensitivity disorders of the immune system that are triggered by an allergen and are characterized by at least one of the symptoms selected from the group of swelling, redness, itching, and irritation of the conjunctiva. Several of the symptoms of allergic conjunctivitis are mediated by the activation of mast cells and basophils in a subject (e.g., in a conjunctiva of the subject) that release several mediators into the tissue of the subject (e.g., one or more mediators selected from the group consisting of: serine proteases, histamine, serotonin, heparin, thromboxane, prostaglandin D2, leukotriene C4, platelet-activating factor, and eosinophil-activating factor). Non-limiting examples of allergic conjunctivitis are atopic keratoconjunctivitis, vernal keratoconjunctivitis, seasonal allergic conjunctivitis, perennial allergic conjunctivitis, and giant papillary conjunctivitis (GPC).

Allergic conjunctivitis can be diagnosed in a subject by assessing one or more (e.g., two, three, or four) symptoms selected from the group of: increased numbers of one or more different types of immune cells (e.g., any of the types of immune cells described herein) in conjunctiva, conjunctival inflammation, conjunctival swelling, conjunctival irritation, eye itchiness, and tearing. In some embodiments of any of the methods described herein, the subject has been diagnosed as having allergic conjunctivitis (e.g., any of the exemplary types of allergic conjunctivitis described herein or known in the art). In some embodiments of any of the methods described herein, the subject has or is suspected of having allergic conjunctivitis (e.g., any of the exemplary types of allergic conjunctivitis described herein or known in the art). In some embodiments of any the methods described herein, the subject has previously received a treatment for allergic conjunctivitis (e.g., a topical steroid), and the subject was not responsive or had a poor response to the treatment. In cases with poor response to conventional anti-allergic medications, especially in cases with chronic disease such as AKC and VKC, significant complications can ensue. There are limited number of available treatments for such refractory cases.

Any of the methods described herein can be used to treat a conjunctival disease, such as allergic conjunctivitis. Any of the methods described herein can result in a decrease in the number of immune cells (e.g., any of the exemplary immune cells described herein) in a conjunctiva in a subject having allergic conjunctivitis and/or can result in a decrease in the migration of immune cells (e.g., any of the exemplary immune cells described herein) in a conjunctiva in a subject having allergic conjunctivitis (e.g., any of the exemplary types of allergic conjunctivitis described herein or known in the art). By reducing the number of immune cells, the methods described herein can reduce the number of symptoms and/or reduce the severity or frequency of symptoms experienced by subjects having allergic conjunctivitis (e.g., refractory allergic conjunctivitis). In addition, by reducing the number and proliferation of conjunctival fibroblasts, any of the methods described herein can reduce the conjunctival scarring which can accompany cases of chronic allergic conjunctivitis.

### Dry Eye Disease

Dry eye disease is a multifactoral disease of the ocular surface and tear film characterized by symptoms of discomfort, visual impairment, and tear film instability. Dry eye syndrome has a variety of different causes. For example, inflammation caused by the infiltration of (or increased numbers of) one or more different types of immune cells (e.g., one or more of any of the different types of immune cells described herein) into the lacrimal glands and conjunctiva can result in a decrease in the secretion of tears produced by these tissues, which can in turn play a role in the pathogenesis of dry eye disease.

Dry eye disease can be diagnosed in a subject by assessing one or more (e.g., two, three, or four) symptoms selected from the group of: dryness, stinging, burning, scratchy sensation in the eye, stringy mucus, increased eye irritation from wind and smoke, eye fatigue after short periods of reading, sensitivity to light, periods of excessive tearing, blurred vision, and red eyes. Additional symptoms of dry eye syndrome are known in the art (see, e.g., Yao et al., Am. J. Med. 124:1016-1018, 2011). Dry eye disease can also be diagnosed using imaging, e.g., using in vivo confocal microscopy. In some embodiments of any of the methods described herein, the subject has been diagnosed as having dry eye disease. In some embodiments of any of the methods described herein, the subject has or is suspected of having dry eye disease. In some embodiments of the methods described herein, the subject has previously received a treatment for a dry eye disease, and the subject was not responsive or had a poor response to the treatment.

Any of the methods described herein can be used to treat a conjunctival disease, such as a dry eye disease. Any of the methods described herein can result in a decrease in the number of immune cells (e.g., any of the exemplary immune cells described herein) in a conjunctiva in a subject having a dry eye disease, and/or can result in a decrease in the migration of immune cells (e.g., any of the exemplary immune cells described herein) in a conjunctiva in a subject having a dry eye disease. By reducing the number or migration of immune cells, which play an important role in pathogenesis of dry eye, any of the methods described herein can improve dry eye disease (e.g., reduce the number of symptoms of dry eye disease and/or reduce the severity and/or frequency of one or more symptoms of dry eye disease in a subject).

### Crosslinking Agents and Compositions Containing a Crosslinking Agent

Crosslinking agents are molecules that can absorb ultraviolet A (UVA) light and are capable of crosslinking collagen protein (e.g., human type I collagen). In the methods described herein, a composition containing a crosslinking agent is topically administered (or subconjunctivally injected) to an eye of the subject (e.g., any of the subjects described herein) and a subsection of conjunctiva in the eye of the subject is irradiated with UVA light. When the UVA light irradiates the subsection of the conjunctiva, the crosslinking agent present in the subsection of the conjunctiva can, for e.g., absorb the UVA light and generate a radical species of the crosslinking agent, which in turn reacts with oxygen (O₂) to generate one or more reactive oxygen species which can, in turn, mediate cross-linking of collagen present in the conjunctiva of the treated eye. Non-limiting examples of crosslinking agents include riboflavin and rose bengal. Another crosslinking agent is diazopyrovoyl PAMAM (8G.1 DAP, 1.3) (shown below).

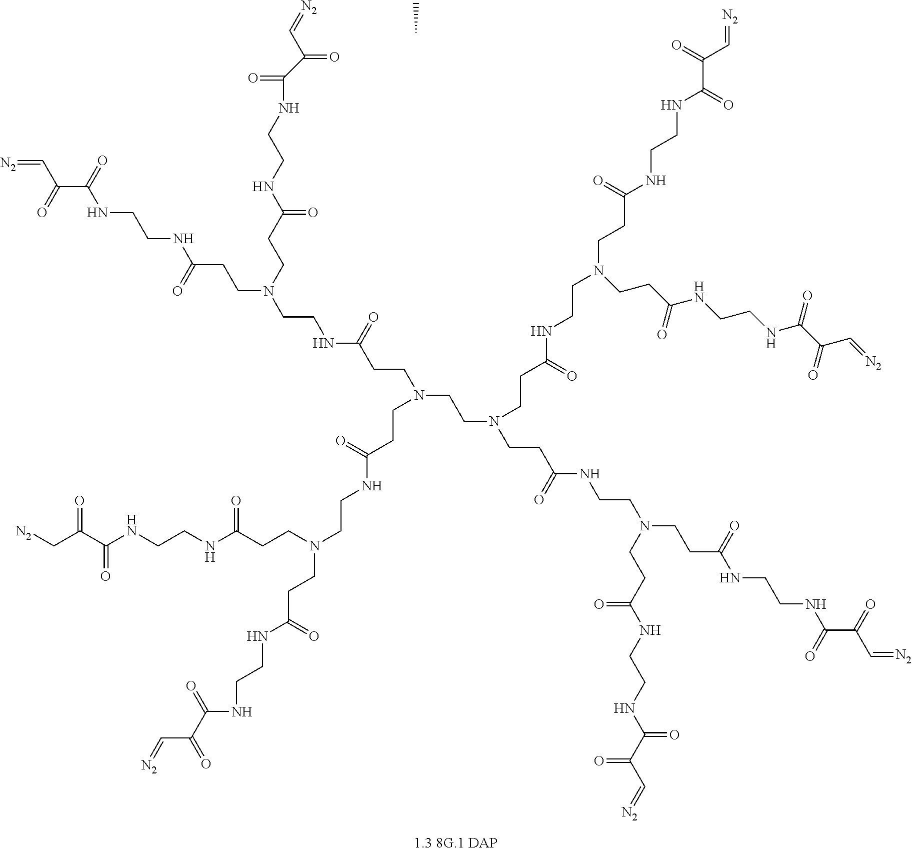

Additional exemplary crosslinking agents are 1,8-naphthalimide dyes (e.g., the 1,8-naphthalimide dyes described in Judy et al. (Proc. SPIE Int. Soc. Opt. Eng. 1882:305-308, 1993)). Other exemplary crosslinking agents are described in Timberlake et al. (Invest. Ophthalmol. Vis. Sci. 38:S510, 1997), Pedone et al. (React. Funct. Polym.66(1): 167-176, 2006), Goodfellow et al. (Biochemistry 28:6346-6360, 1989), Mosier et al. (J. Org. Chem. 60(21):6953-6958, 1995), and Givens et al. (Photochem. Photobiol. 78(1):23-29, 2003). Another exemplary crosslinking agent is the rose bengal diisopropyl amine derivative described in Pitts et al. (Photochem. Photobiol. 76(2):135-144, 2002). Additional examples of crosslinking agents that can be used in the present methods are known in the art.

Additional crosslinking agents that can be used in the present methods can be identified by performing additional in vivo or in vitro assays. For example, different crosslinking agents can be added to a composition containing collagen or a composition containing conjunctival cells and irradiated with UVA light (e.g., using any combination of the exemplary parameters described herein). Successful collagen crosslinking can be identified in the composition containing collagen using any of the exemplary methods described in the Examples (e.g., confocal microscopy, 2-photon microscopy, optical coherence tomography, and protease digestion). Successful collagen crosslinking in a composition containing conjunctival cells (an adherent cell line) can be determined using any of the exemplary methods described in the Examples (e.g., confocal microscopy, transmission electron microscopy, 2-photon microscopy, and optical coherence tomography). Successful collagen crosslinking can also be assessed in an animal model (e.g., any of the exemplary animal models described in the Examples).

The methods described herein include the topical administration (or subconjunctival injection) of a composition containing a crosslinking agent to an eye of the subject. A crosslinking agent can have a final concentration of between 0.01% and 5.0% (e.g., between 0.01% and 4.0%, between 0.01% and 3.5%, between 0.01% and 3.0%, between 0.01% and 2.5%, between 0.01% and 2.0%, between 0.01% and 1.5%, between 0.01% and 1.0%, between 0.05% and 1.0%, between 0.08% and 0.8%, between 0.08% and 0.5%, and between 0.1% and 0.5%) weight/volume or weight/weight in any of the compositions containing a crosslinking agent described herein. The compositions containing a crosslinking agent can contain one or more (e.g., two, three, or four) different crosslinking agents. The one or more different crosslinking agents can each be present in the composition at a final concentration of between 0.01% and 5.0% (e.g., between 0.01% and 4.0%, between 0.01% and 3.5%, between 0.01% and 3.0%, between 0.01% and 2.5%, between 0.01% and 2.0%, between 0.01% and 1.5%, between 0.01% and 1.0%, between 0.05% and 1.0%, between 0.08% and 0.8%, between 0.08% and 0.5%, and between 0.1% and 0.5%) weight/volume or weight/weight. The composition can also contain one or more (e.g., two, three, four, five, six, seven, eight, nine, or ten) of the following: one or more (e.g., two, three, or four) antibiotics (e.g., neomycin, sulfacetamide sodium, erythromycin, silver sulfadiazine, retapmulin, mupirocin, bacitracin, neomycin, polymyxin B, pramoxine, and mupirocin), one or more (e.g., two, three, or four) anti-fungal agents (e.g., benzoic acid, salicyclic acid, undecylenic acid, ketoconazole, nystatin, tolnaftate, miconazole, econazole, miconazole, zinc oxide, ciclopirox, ciclopirox, sertaconazole, terbinafine, and clotrimazole), one or more (e.g., two, three, or four) analgesics and/or numbing agents (e.g., lidocaine, benzocaine, butamben, dibucaine, oxybuprocaine, pramoxine, proparacaine, proxymetacaine, and tetracaine), one or more (e.g., two, three, or four) lubricants (e.g., artificial tears or serum tears), a physiologically acceptable buffer (e.g., saline), and/or one or more (e.g., two, three, or four) anti-inflammatory agents (e.g., a steroid, bromfenac, cyclosporine, nepafenac, ketorolac, flurbiprofen, suprofen, diclofenac, and bromfenac). Any of the compositions described herein can also contain one or more other agents selected from the group of metal chelators (e.g., ethylenediaminetetraacetic acid and ethylene glycol tetraacetic acid), benzalkonium chloride, or alcohol. Any of the compositions described herein can also a final concentration of dextran (e.g., recombinant human dextran) between 5% and 30% (e.g., between 5% and 10%, between 10% and 15%, between 15% and 20%, between 20% and 25%, between 25% and 30%, between 5% and 25%, between 10% and 25%, between 15% and 25%, and between 15% and 20%) weight/volume or weight/weight. Any of the compositions containing a crosslinking agent can be isotonic (e.g., allow for the swelling of the conjunctiva of the subject following administration). Non-limiting examples of the compositions described herein include the following solutions available from Avedro (Waltham, Mass.): 0.1% riboflavin and 20% dextran, isotonic; 0.25% riboflavin, saline, isotonic; 0.1% riboflavin, saline, hydroxypropyl methylcellulose (HPMC); and 0.25% riboflavin, HPMC, benzalkonium chloride (BAC), ethylenediaminetetraacetic acid (EDTA), and tris(hydroxymethyl)aminomethane (TRIS).

In some embodiments of any of the methods described herein, the composition containing the crosslinking agent is administered (e.g., by topical administration or subconjunctival injection) one or more (e.g., two, three, four, five, six, seven, eight, nine, or ten) times prior to the irradiation with UVA light, one or more (e.g., two, three, four, five, six, seven, eight, nine, or ten) times prior to and one or more (e.g., two, three, four, five, six, seven, eight, nine, or ten) times intermittently (e.g., periodically topically administered to the eye every 1 minute to about 5 minutes) during the irradiation with UVA light, or one or more (e.g., two, three, four, five, six, seven, eight, nine, or ten) times intermittently during the irradiation with UVA light.

### UVA Light Source and Exposure

The methods described include irradiating a subsection of a conjunctiva in an eye in a subject after a composition containing a crosslinking agent has been topically administered (or subconjunctivally injected) to the eye. In any of the methods described herein, the irradiation can occur over a period of less than 2 hours (e.g., less than 90 minutes, less than 80 minutes, less than 70 minutes, less than 60 minutes, less than 55 minutes, less than 50 minutes, less than 45 minutes, less than 40 minutes, less than 35 minutes, less than 30 minutes, less than 25 minutes, less than 20 minutes, less than 15 minutes, less than 10 minutes, less than 8 minutes, less than 6 minutes, less than 4 minutes, less than 3 minutes, less than 2 minutes, or less than 1 minute). In any of the methods described herein, the UVA light can have a wavelength of between 315 nm to 400 nm (e.g., between 340 nm to 375 nm, between 360 nm and 370 nm, between 330 nm and 385 nm, between 350 nm and 380 nm, between 315 nm and 350 nm, between 320 nm and 355 nm, between 325 nm and 360 nm, between 330 nm and 365 nm, between 335 nm and 370 nm, between 340 nm and 375 nm, between 345 nm and 380 nm, between 350 nm and 385 nm, between 355 nm and 390 nm, between 360 nm and 395 nm, and between 365 and 400 nm). In some embodiments of any of the methods described herein, the UVA light has a wavelength of about 365 nm.

In any of the methods described herein, the irradiance of the UVA light is between 0.1 mW/cm² and 45 mW/cm² (e.g., 0.1 mW/cm² and 40 mW/cm², between 0.1 mW/cm² and 35 mW/cm², 0.1 mW/cm² and 30 mW/cm², between 0.1 mW/cm² and 25 mW/cm², between 0.1 mW/cm² and 20 mW/cm², between 0.1 mW/cm² and 15 mW/cm², between 0.1 mW/cm² and 12 mW/cm², between 0.1 mW/cm² and 10 mW/cm², between 0.1 mW/cm² and 8 mW/cm², between 2 mW/cm² and 7 mW/cm², between 3 mW/cm² and 6 mW/cm², between 1 mW/cm² and 5 mW/cm², between 5 mW/cm² and 10 mW/cm², between 10 mW/cm² and 15 mW/cm², between 1 mW/cm² and 4 mW/cm², between 2 mW/cm² and 5 mW/cm², between 3 mW/cm² and 6 mW/cm², between 4 mW/cm² and 7 mW/cm², between 5 mW/cm² and 8 mW/cm², between 6 mW/cm² and 9 mW/cm², and between 7 mW/cm² and 10 mW/cm²). In any of the methods described herein, the irradiating provides a dose of between 0.1 J/cm² to 15 J/cm² (e.g., between about 2 J/cm² and 14 J/cm², between 2 J/cm² and 13 J/cm², between 2 J/cm² and 12 J/cm², between 2 J/cm² and 11 J/cm², between 2 J/cm² and 10 J/cm², between 2 J/cm² and 9 J/cm², between 2 J/cm² and 8 J/cm², between 2 J/cm² and 7 J/cm², between 2 J/cm² and 6 J/cm², between 3 J/cm² and 8 J/cm², between 4 J/cm² and 9 J/cm², between 5 J/cm² and 10 J/cm², between 6 J/cm² and 11 J/cm², between 7 J/cm² and 12 J/cm², between 8 J/cm² and 13 J/cm², between 9 J/cm² and 14 J/cm², between 10 J/cm² and 15 J/cm², and between 2 J/cm² and 5 J/cm²). In any of the methods described herein, the subsection of the conjunctiva irradiated has an area of between 3 mm² and 300 mm² (e.g., between 3 mm² and 250 mm², between 3 mm² and 200 mm², between 3 mm² and 150 mm², between 3 mm² and 100 mm², between 3 mm² and 50 mm², between 3 mm² and 25 mm², between 3 mm² and 20 mm², between 3 mm² and 15 mm², between 3 mm² and 10 mm², between 10 mm² and 20 mm², and between 20 mm² and 30 mm²).

In any of the embodiments described herein, the UVA light that is used to irradiate a subsection of the conjunctiva is pulsed UVA light. For example, the UVA light may alternate for between being turned on for a period of between about 0.01 second to about 10 seconds (e.g., between about 0.01 second to about 8 seconds, between about 0.01 second to about 6 seconds, between about 0.01 second to about 4 seconds, between about 0.01 second to about 3 seconds, between about 0.01 second to about 2 seconds, or between about 0.01 second to about 1 second) and being turned off for a period of between about 0.01 second to about 50 seconds (e.g., between about 0.01 second and about 45 seconds, between about 0.01 second and about 40 seconds, between about 0.01 second and about 35 seconds, between about 0.01 second and about 30 seconds, between about 0.01 second and about 25 seconds, between about 0.01 second and about 20 seconds, between about 0.01 second and about 15 seconds, between about 0.01 second and 10 seconds, between about 0.01 second and about 5 seconds, between about 0.01 second and about 3 seconds, between about 0.01 second and about 2 seconds, or between about 0.01 second and about 1 second).

A variety of devices that emit UVA light can be used to irradiate a subsection of the conjunctiva (e.g., irradiate a subsection of the conjunctiva as described in any of the methods described herein). For example, a pair of specialized goggles with light-emitting diode (LED) lights can be used to irradiate a subsection of a conjunctiva in the eye of a subject. UVA light can also be produced using a laser that emits UVA light (e.g., UV gas lasers, laser diodes, a double diode laser, and UV solid-state lasers). Additional sources of UVA light are known in the art.

In some embodiments of any of the methods described herein, the UVA light does not penetrate the conjunctiva to a depth of greater than 250 from the surface of the conjunctiva (e.g., penetrates the conjunctiva to a depth of no greater than 200 μm, no greater than 150 μm, no greater than 100 μm, no greater than 50 μm, or no greater than 25 μm from the surface of the conjunctiva). In some embodiments, the UVA light does not irradiate the sclera, limbus, and/or cornea. This can be achieved by using a screen or filter than prevents or blocks the UVA light from irradiating the sclera (while allowing the UVA light to irradiate a subsection of the conjunctiva).

### Methods of Treating a Conjunctival Disease

Provided herein are methods of treating a conjunctival disease. These methods include topically administering (or subconjunctivally injecting) a composition containing a crosslinking agent (e.g., any of the compositions containing a crosslinking agent described herein or known in the art) into an eye of a subject having or suspected of having a conjunctival disease; and irradiating a subsection of the conjunctiva of the eye of the subject with ultraviolet A (UVA) light for a time and duration sufficient to treat the conjunctival disease in the eye of the subject. The conjunctival diseases that can be treated using these methods include without limitation: conjunctivochalasis, conjunctival bleb thinning after a glaucoma surgical procedure, pterygium, a cicatricial conjunctival disorder, autoimmune disease of conjunctiva, allergic conjunctivitis, and dry eye disease.

In some embodiments of these methods, the subject has or is suspected of having a conjunctival disease, e.g., conjunctivochalasis, conjunctival bleb thinning after a glaucoma surgical procedure, pterygium, a cicatricial conjunctival disorder, autoimmune disease of conjunctiva, allergic conjunctivitis, or dry eye disease. In some embodiments of these methods, the subject is diagnosed as having a conjunctival disorder, e.g., conjunctivochalasis, conjunctival bleb thinning after a glaucoma surgical procedure, pterygium, a cicatricial conjunctival disorder, autoimmune disease of conjunctiva, allergic conjunctivitis, and dry eye disease. Non-limiting methods of diagnosing a subject as having a conjunctival disease (e.g., conjunctivochalasis, conjunctival bleb thinning after by a glaucoma surgical procedure, pterygium, a cicatricial conjunctival disorder, autoimmune disease of conjunctiva, allergic conjunctivitis, or dry eye disease) are described herein. In some embodiments of these methods, a subject may present with two or more symptoms (e.g., three, four, five, or six) symptoms of a conjunctival disease. In some embodiments, the subject may have an early stage (or a mild or moderate form) of a conjunctival disease, or may have two or more (e.g., three, four, five, or six) mild symptoms of a conjunctival disease. In other embodiments, the subject may have a late stage (or severe form) of a conjunctival disease. In some embodiments, the subject may have already received treatment for a conjunctival disease, and the subject was determined to be nonresponsive or show a poor response to the prior treatment.

Any combination of the exemplary features of UVA light irradiation (e.g., wavelengths, irradiances, periods of irradiation, UVA light doses, and/or areas of the subsection of conjunctiva irradiated) can be used in these methods. In addition, a pulsed UVA light can also be used in these methods (e.g., pulsed UVA light having any of the exemplary parameters described herein).

Any of the exemplary crosslinking agents and exemplary compositions containing a crosslinking agent described herein can be used in these methods. In some embodiments, a subject is topically administered more than one dose (e.g., two, three, four, five, six, seven, eight, nine, or ten doses) of a composition containing a crosslinking agent (e.g., according to any of the exemplary administration schedules described herein). In some embodiments, a subject is instructed to self-administer to the eye a composition containing a crosslinking agent (e.g., instructed to self-administer eye drops, e.g., every one to five minutes, that contain a crosslinking agent). In some embodiments, the composition containing a crosslinking agent is subconjunctivally injected into the subject. In some embodiments, the composition containing a crosslinking agent is a hypotonic solution that is sufficient to swell the conjunctiva in the eye that is administered the composition. In some embodiments, the composition containing a crosslinking agent is a pharmaceutically acceptable solution containing about 0.1% riboflavin (w/w or w/v) and about 20% dextran (w/w or w/v).

In some embodiments, the irradiating occurs at a period of between 5 seconds and 2 hours after the administration of a composition containing a crosslinking agent, or between 5 seconds and 2 hours after the last administration of a composition containing a crosslinking agent (when more than one administration of a composition containing a crosslinking agent occurs). In some embodiments, the composition containing a crosslinking agent is administered one or more times (e.g., two, three, four, five, six, seven, eight, nine, or ten times) prior the irradiation with UVA light. In some embodiments, the composition containing a crosslinking agent is administered one or more times (e.g., two, three, four, five, six, seven, eight, nine, or ten times) prior to the irradiation with UVA light and administered one or more times (e.g., two, three, four, five, six, seven, eight, nine, or ten times), intermittently, during the UVA light irradiation. In some embodiments, the composition containing a crosslinking agent is administered one or more times (e.g., two, three, four, five, six, seven, eight, nine, or ten times) during the UVA light irradiation.

Some embodiments further include administering (e.g., topically administering or subconjunctivally injecting) a hypotonic solution that allows for the swelling of the conjunctiva in the eye to which the hypotonic solution is administered. In some embodiments, the hypotonic solution is administered one or more times (e.g., two, three, four, five, six, seven, eight, nine, or ten times) during any one or more periods of the treatment: one or more times (e.g., two, three, four, five, six, seven, eight, nine, or ten times) prior to administration of the composition containing a crosslinking agent, one or more times (e.g., two, three, four, five, six, seven, eight, nine, or ten times) intermittently during the administration of the composition containing a crosslinking agent, and one or more times (e.g., two, three, four, five, six, seven, eight, nine, or ten times) intermittently during the UVA light irradiation. In other embodiments, the hypotonic solution is administered one or more times (e.g., two, three, four, five, six, seven, eight, nine, or ten times) after the administration of the composition containing a crosslinking agent, but before the irradiation with UVA light. In some embodiments, the irradiation with UVA light occurs between 1 minutes and 2 hours (e.g., between 1 minute and 1 hour, between 1 minute and 45 minutes, between 1 minute and 30 minutes, between 1 minute and 20 minutes, and between 1 minute and 10 minutes) after the administration of the hypotonic solution or the last administration of the hypotonic solution (in instances where the hypotonic solution is administered more than once to the eye of the subject). In some embodiments of any of these methods, the composition containing the crosslinking agent and the hypotonic solution can be the same solution (e.g., a hypotonic solution containing a crosslinking agent, e.g., a riboflavin solution without containing dextran, e.g., VibeX Rapid from Avedro).

These methods can be performed by any medical professional (e.g., a physician, a nurse, a technician, or a physician's assistant). These methods can be performed on a subject more than once (e.g., two, three, four, five, or six times). In examples where the methods are performed more than once, the method can be performed with a frequency of at least once every five years (e.g., once every four years, once every three years, once every two years, once every year, or once every six months). A physician can readily determine the frequency at which the methods described herein should be performed on a subject (e.g., based on the severity, frequency, and number of symptoms of a conjunctival disease observed in the subject).

The efficacy of the present methods in a subject can be determined by observing the severity, frequency, and number of symptoms of a conjunctival disease experienced by the subject. For example, successful treatment will result in a decrease in the severity, frequency, and/or number of symptoms (e.g., any of the exemplary symptoms described here or known in the art) experienced by a subject having a conjunctival disease. For some conjunctival diseases, effective treatment can also be observed by a detection of an increase in the rigidity of the conjunctiva (e.g., as observed using slit lamp examination, in vivo confocal microscopy, 2-photon microscopy, and optical coherence tomography, or any of the exemplary methods described in the Examples), a decrease in the proliferation or number conjunctival fibroblasts (e.g., as detected by in vivo confocal microscopy), or a decrease in the number of immune cells present in the conjunctiva (e.g., as detecting using in vivo confocal microscopy) e.g., as compared to the same measured parameter in a subject prior to treatment.

### Methods of Increasing the Rigidity of a Conjunctiva

Provided herein are methods of increasing the rigidity of a conjunctiva in an eye of a subject. These methods include topically administering (or conjunctivally injecting) a composition containing a crosslinking agent (e.g., any of the compositions containing a crosslinking agent described herein or known in the art) into an eye of a subject having or suspected of having decreased rigidity in a conjunctiva of the eye; and irradiating a subsection of the conjunctiva of the eye of the subject with ultraviolet A (UVA) light for a time and duration sufficient to increase the rigidity of the conjunctiva in the eye of the subject (e.g., as compared to the rigidity of the conjunctiva in the eye of the subject prior to treatment).

In some embodiments of these methods, the subject has or is suspected of having a conjunctival disease, e.g., conjunctivochalasis or conjunctival bleb thinning after a glaucoma surgical procedure (e.g., trabeculectomy). In some embodiments of these methods, the subject is diagnosed as having a conjunctival disorder, e.g., conjunctivochalasis or conjunctival bleb thinning after a glaucoma surgical procedure (e.g., trabeculectomy). Non-limiting methods of diagnosing a subject as having a conjunctival disease (e.g., conjunctivochalasis or conjunctival bleb thinning after a glaucoma surgical procedure) are described herein. In some embodiments of these methods, a subject may present with two or more symptoms (e.g., three, four, five, or six) symptoms of a conjunctival disease (e.g., conjunctivochalasis or conjunctival bleb thinning after a glaucoma surgical procedure). In some embodiments, the subject may have an early stage (or a mild or moderate form) of a conjunctival disease (e.g., conjunctivochalasis or conjunctival bleb thinning after a glaucoma surgical procedure), or may have two or more mild symptoms of a conjunctival disease (e.g., conjunctivochalasis or conjunctival bleb thinning after a glaucoma surgical procedure). In other embodiments, the subject may have a late stage (or severe form) of a conjunctival disease (e.g., conjunctivochalasis or conjunctival bleb thinning after a glaucoma surgical procedure). In some embodiments, the subject may have already received treatment for a conjunctival disease, and the subject was determined to be nonresponsive or show a poor response to the prior treatment. In some embodiments, the subject has already had a glaucoma surgical procedure (e.g., a trabeculectomy), or a trabeculectomy was performed on the subject within the last year (e.g., within the last 10 months, 8 months, 6 months, 4 months, 2 months, or 1 month).

Any combination of the exemplary features of UVA light irradiation (e.g., wavelengths, irradiances, periods of irradiation, UVA light doses, and/or areas of the subsection of conjunctiva irradiated) can be used in these methods. In addition, a pulsed UVA light can also be used in these methods (e.g., pulsed UVA light having any of the exemplary parameters described herein).

Any of the exemplary crosslinking agents and exemplary compositions containing a crosslinking agent described herein can be used in these methods. In some embodiments, a subject is topically administered more than one dose (e.g., two, three, four, five, six, seven, eight, nine, or ten doses) of a composition containing a crosslinking agent (e.g., according to any of the exemplary administration schedules described herein). In some embodiments, a subject is instructed to self-administer to the eye a composition containing a crosslinking agent (e.g., instructed to self-administer eye drops, e.g., every one to five minutes, that contain a crosslinking agent). In some embodiments, the composition containing a crosslinking agent is subconjunctivally administered to the subject. In some embodiments, the composition containing a crosslinking agent is a hypotonic solution that is sufficient to swell the conjunctiva in the eye that is administered the composition. In some embodiments, the composition containing a crosslinking agent is a pharmaceutically acceptable solution containing about 0.1% riboflavin (w/w or w/v) and about 20% dextran (w/w or w/v).

In some embodiments, the irradiating occurs at a period of between 5 seconds and 2 hours after the administration of a composition containing a crosslinking agent, or between 5 seconds and 2 hours after the last administration of a composition containing a crosslinking agent (when more than one administration of a composition containing a crosslinking agent occurs). In some embodiments, the composition containing a crosslinking agent is administered one or more times (e.g., two, three, four, five, six, seven, eight, nine, or ten times) prior the irradiation with UVA light. In some embodiments, the composition containing a crosslinking agent is administered one or more times (e.g., two, three, four, five, six, seven, eight, nine, or ten times) prior to the irradiation with UVA light and administered one or more times (e.g., two, three, four, five, six, seven, eight, nine, or ten times), intermittently, during the UVA light irradiation. In some embodiments, the composition containing a crosslinking agent is administered one or more times (e.g., two, three, four, five, six, seven, eight, nine, or ten times) during the UVA light irradiation.

Some embodiments further include administering (e.g., topically administering or subconjunctivally injecting) a hypotonic solution that allows for the swelling of the conjunctiva in the eye to which the hypotonic solution is administered. In some embodiments, the hypotonic solution is administered one or more times (e.g., two, three, four, five, six, seven, eight, nine, or ten times) during any one or more periods of the treatment: one or more times (e.g., two, three, four, five, six, seven, eight, nine, or ten times) prior to administration of the composition containing a crosslinking agent, one or more times (e.g., two, three, four, five, six, seven, eight, nine, or ten times) intermittently during the administration of the composition containing a crosslinking agent, and one or more times (e.g., two, three, four, five, six, seven, eight, nine, or ten times) intermittently during the UVA light irradiation. In other embodiments, the hypotonic solution is administered one or more times (e.g., two, three, four, five, six, seven, eight, nine, or ten times) after the administration of the composition containing a crosslinking agent, but before the irradiation with UVA light. In some embodiments, the irradiation with UVA light occurs between 1 minutes and 2 hours (e.g., between 1 minute and 1 hour, between 1 minute and 45 minutes, between 1 minute and 30 minutes, between 1 minute and 20 minutes, and between 1 minute and 10 minutes) after the administration of the hypotonic solution or the last administration of the hypotonic solution (in instances where the hypotonic solution is administered more than once to the eye of the subject). In some embodiments of any of these methods, the composition containing the crosslinking agent and the hypotonic solution can be the same solution (e.g., a hypotonic solution containing a crosslinking agent).

These methods can be performed by any medical professional (e.g., a physician, a nurse, a technician, or a physician's assistant). These methods can be performed on a subject more than once (e.g., two, three, four, five, or six times). In examples where the methods are performed more than once, the method can be performed with a frequency of at least once every five years (e.g., once every four years, once every three years, once every two years, once every year, or once every six months). A physician can readily determine the frequency at which the methods described herein should be performed on a subject (e.g., based on the severity, frequency, and number of symptoms of a conjunctival disease (e.g., conjunctivochalasis or conjunctival bleb thinning caused by a glaucoma surgical procedure observed in the subject).

The efficacy of the present methods in a subject can be determined by observing the severity, frequency, and number of symptoms of a conjunctival disease (e.g., conjunctivochalasis or conjunctival bleb thinning after a glaucoma surgical procedure) experienced by the subject. For example, successful treatment will result in a decrease in the severity, frequency, and/or number of symptoms (e.g., any of the exemplary symptoms described here or known in the art) experienced by a subject having a conjunctival disorder (e.g., conjunctivochalasis or conjunctival bleb thinning after a glaucoma surgical procedure). In some of these methods, effective treatment can also be observed by a detection of an increase in the rigidity of the conjunctiva (e.g., decreased conjunctival looseness as observed using slit lamp examination, increased thickness of bleb wall by optical coherence tomography, improved collagen architecture by in vivo confocal microscopy and 2-photon microscopy, or any of the exemplary methods described in the Examples), e.g., as compared to the same measured parameter in a subject prior to treatment. In some of these methods, effective treatment can also be observed by clinical examination (e.g., biomechanical examination) of the subject's eye.

### Methods of Decreasing the Proliferation or Number of Conjunctival Fibroblasts

Provided herein are methods of decreasing the proliferation or number of conjunctival fibroblasts in a conjunctiva in a subject. These methods include topically administering (or subconjunctivally injecting) a composition containing a crosslinking agent (e.g., any of the compositions containing a crosslinking agent described herein or known in the art) into an eye of a subject having or suspected of having increased proliferation or number of conjunctival fibroblasts in a conjunctiva of the eye; and irradiating a subsection of the conjunctiva of the eye of the subject with ultraviolet A (UVA) light for a time and duration sufficient to decrease the proliferation or number of conjunctival fibroblasts in the subsection of the conjunctiva in the eye of the subject.

In some embodiments of these methods, the subject has or is suspected of having a conjunctival disease, e.g., pterygium or a cicatricial conjunctival disorder (e.g., any of the cicatricial conjunctival disorders described herein or known in the art). In some embodiments of these methods, the subject is diagnosed as having a conjunctival disorder, e.g., pterygium or a cicatricial conjunctival disorder (e.g., any of the cicatricial conjunctival disorders described herein or known in the art). Non-limiting methods of diagnosing a subject as having a conjunctival disease (e.g., pterygium or a cicatricial conjunctival disorder) are described herein. In some embodiments of these methods, a subject may present with two or more symptoms (e.g., three, four, five, or six) symptoms of a conjunctival disease (e.g., any of the exemplary symptoms of pterygium or a cicatricial conjunctival disorder described herein). In some embodiments, the subject may have an early stage (or a mild or moderate form) of a conjunctival disease (e.g., pterygium or a cicatricial conjunctival disorder), or may have two or more mild symptoms of a conjunctival disease (e.g., pterygium or a cicatricial conjunctival disorder). In other embodiments, the subject may have a late stage (or severe form) of a conjunctival disease (e.g., pterygium or a cicatricial conjunctival disorder). In some embodiments, the subject may have already received treatment for a conjunctival disease, and the subject was determined to be nonresponsive or show a poor response to the prior treatment.

Any combination of the exemplary features of UVA light irradiation (e.g., wavelengths, irradiances, periods of irradiation, UVA light doses, and/or areas of the subsection of conjunctiva irradiated) can be used in these methods. In addition, a pulsed UVA light can also be used in these methods (e.g., pulsed UVA light having any of the exemplary parameters described herein).

Any of the exemplary crosslinking agents and exemplary compositions containing a crosslinking agent described herein can be used in these methods. In some embodiments, a subject is topically administered more than one dose (e.g., two, three, four, five, six, seven, eight, nine, or ten doses) of a composition containing a crosslinking agent (e.g., according to any of the exemplary administration schedules described herein). In some embodiments, a subject is instructed to self-administer to the eye a composition containing a crosslinking agent (e.g., instructed to self-administer eye drops, e.g., every one to five minutes, that contain a crosslinking agent). In some embodiments, the composition containing a crosslinking agent is subconjunctivally injected into the subject. In some embodiments, the composition containing a crosslinking agent is a hypotonic solution that is sufficient to swell the conjunctiva in the eye that is administered the composition. In some embodiments, the composition containing a crosslinking agent is a pharmaceutically acceptable solution containing about 0.1% riboflavin (w/w or w/v) and about 20% dextran (w/w or w/v).

In some embodiments, the irradiating occurs at a period of between 5 seconds and 2 hours after the administration of a composition containing a crosslinking agent, or between 5 seconds and 2 hours after the last administration of a composition containing a crosslinking agent (when more than one administration of a composition containing a crosslinking agent occurs). In some embodiments, the composition containing a crosslinking agent is administered one or more times (e.g., two, three, four, five, six, seven, eight, nine, or ten times) prior the irradiation with UVA light. In some embodiments, the composition containing a crosslinking agent is administered one or more times (e.g., two, three, four, five, six, seven, eight, nine, or ten times) prior to the irradiation with UVA light and administered one or more times (e.g., two, three, four, five, six, seven, eight, nine, or ten times), intermittently, during the UVA light irradiation. In some embodiments, the composition containing a crosslinking agent is administered one or more times (e.g., two, three, four, five, six, seven, eight, nine, or ten times) during the UVA light irradiation.

Some embodiments further include administering (e.g., topically administering or subconjunctivally injecting) a hypotonic solution that allows for the swelling of the conjunctiva in the eye to which the hypotonic solution is administered. In some embodiments, the hypotonic solution is administered one or more times (e.g., two, three, four, five, six, seven, eight, nine, or ten times) during any one or more periods of the treatment: one or more times (e.g., two, three, four, five, six, seven, eight, nine, or ten times) prior to administration of the composition containing a crosslinking agent, one or more times (e.g., two, three, four, five, six, seven, eight, nine, or ten times) intermittently during the administration of the composition containing a crosslinking agent, and one or more times (e.g., two, three, four, five, six, seven, eight, nine, or ten times) intermittently during the UVA light irradiation. In other embodiments, the hypotonic solution is administered one or more times (e.g., two, three, four, five, six, seven, eight, nine, or ten times) after the administration of the composition containing a crosslinking agent, but before the irradiation with UVA light. In some embodiments, the irradiation with UVA light occurs between 1 minutes and 2 hours (e.g., between 1 minute and 1 hour, between 1 minute and 45 minutes, between 1 minute and 30 minutes, between 1 minute and 20 minutes, and between 1 minute and 10 minutes) after the administration of the hypotonic solution or the last administration of the hypotonic solution (in instances where the hypotonic solution is administered more than once to the eye of the subject). In some embodiments of any of these methods, the composition containing the crosslinking agent and the hypotonic solution can be the same solution (e.g., a hypotonic solution containing a crosslinking agent).

These methods can be performed by any medical professional (e.g., a physician, a nurse, a technician, or a physician's assistant). These methods can be performed on a subject more than once (e.g., two, three, four, five, or six times). In examples where the methods are performed more than once, the method can be performed with a frequency of at least once every five years (e.g., once every four years, once every three years, once every two years, once every year, or once every six months). A physician can readily determine the frequency at which the methods described herein should be performed on a subject (e.g., based on the severity, frequency, and number of symptoms of a conjunctival disease (e.g., pterygium or a conjunctival cicatricial disorder) observed in the subject, or the regrowth of conjunctival fibroblasts following surgical resection of fibrovascular tissue formed over the cornea in a subject having pterygium).

The efficacy of the present methods in a subject can be determined by observing the severity, frequency, and number of symptoms of a conjunctival disease (e.g., pterygium or a cicatricial conjunctival disorder) experienced by the subject. For example, successful treatment will result in a decrease in the severity, frequency, and/or number of symptoms (e.g., any of the exemplary symptoms described here or known in the art) experienced by a subject having a conjunctival disorder (e.g., pterygium or a cicatricial conjunctival disorder). In some of these methods, effective treatment can also be observed by a detection of an decrease in the proliferation or number of conjunctival fibroblasts in the treated eye of the subject (e.g., regression or lack of progression of pterygium or a conjunctival cicatricial disease, a reduction in conjunctival fibrovascular proliferation, conjunctival scar, or symblepharon formation, as, e.g., observed using slit lamp examination, in vivo confocal microscopy, 2-photon microscopy, or optical coherence tomography, or any of the other methods described in the Examples), e.g., as compared to the same measured parameter in a subject prior to treatment.

### Methods of Decreasing the Number of Immune Cells in a Conjunctiva

Provided herein are methods of decreasing the number of immune cells in a conjunctiva in a subject. These methods include topically administering (or subconjunctivally injecting) a composition containing a crosslinking agent (e.g., any of the compositions containing a crosslinking agent described herein or known in the art) into an eye of a subject having or suspected of having an increased number of immune cells in a conjunctiva of the eye; and irradiating a subsection of the conjunctiva of the eye of the subject with ultraviolet A (UVA) light for a time and duration sufficient to reduce the number of immune cells in the subsection of the conjunctiva in the eye of the subject.

In some embodiments of these methods, the subject has or is suspected of having a conjunctival disease, e.g., autoimmune disease of conjunctiva, allergic conjunctivitis, and dry eye disease. In some embodiments of these methods, the subject is diagnosed as having a conjunctival disorder, e.g., autoimmune disease of conjunctiva, allergic conjunctivitis, and dry eye disease. Non-limiting methods of diagnosing a subject as having a conjunctival disease (e.g., autoimmune disease of conjunctiva, allergic conjunctivitis, and dry eye disease) are described herein. In some embodiments of these methods, a subject may present with two or more symptoms (e.g., three, four, five, or six) symptoms of a conjunctival disease (e.g., any of the exemplary symptoms of autoimmune disease of conjunctiva, allergic conjunctivitis, and dry eye disease described herein). In some embodiments, the subject may have an early stage (or a mild or moderate form) of a conjunctival disease (e.g., autoimmune disease of conjunctiva, allergic conjunctivitis, and dry eye disease), or may have two or more mild symptoms of a conjunctival disease (e.g., autoimmune disease of conjunctiva, allergic conjunctivitis, and dry eye disease). In other embodiments, the subject may have a late stage (or severe form) of a conjunctival disease (e.g., autoimmune disease of conjunctiva, allergic conjunctivitis, and dry eye disease). In some embodiments, the subject may have already received treatment for a conjunctival disease, and the subject was determined to be nonresponsive or show a poor response to the prior treatment.

Any combination of the exemplary features of UVA light irradiation (e.g., wavelengths, irradiances, periods of irradiation, UVA light doses, and/or areas of the subsection of conjunctiva irradiated) can be used in these methods. In addition, a pulsed UVA light can also be used in these methods (e.g., pulsed UVA light having any of the exemplary parameters described herein).

Any of the exemplary crosslinking agents and exemplary compositions containing a crosslinking agent described herein can be used in these methods. In some embodiments, a subject is topically administered more than one dose (e.g., two, three, four, five, six, seven, eight, nine, or ten doses) of a composition containing a crosslinking agent (e.g., according to any of the exemplary administration schedules described herein). In some embodiments, a subject is instructed to self-administer to the eye a composition containing a crosslinking agent (e.g., instructed to self-administer eye drops, e.g., every one to five minutes, that contain a crosslinking agent). In some embodiments, the composition containing a crosslinking agent is subconjunctivally injected into the subject. In some embodiments, the composition containing a crosslinking agent is a hypotonic solution that is sufficient to swell the conjunctiva in the eye that is administered the composition. In some embodiments, the composition containing a crosslinking agent is a pharmaceutically acceptable solution containing about 0.1% riboflavin (w/w or w/v) and about 20% dextran (w/w or w/v).

In some embodiments, the irradiating occurs at a period of between 5 seconds and 2 hours after the administration of a composition containing a crosslinking agent, or between 5 seconds and 2 hours after the last administration of a composition containing a crosslinking agent (when more than one administration of a composition containing a crosslinking agent occurs). In some embodiments, the composition containing a crosslinking agent is administered one or more times (e.g., two, three, four, five, six, seven, eight, nine, or ten times) prior the irradiation with UVA light. In some embodiments, the composition containing a crosslinking agent is administered one or more times (e.g., two, three, four, five, six, seven, eight, nine, or ten times) prior to the irradiation with UVA light and administered one or more times (e.g., two, three, four, five, six, seven, eight, nine, or ten times), intermittently, during the UVA light irradiation. In some embodiments, the composition containing a crosslinking agent is administered one or more times (e.g., two, three, four, five, six, seven, eight, nine, or ten times) during the UVA light irradiation.

Some embodiments further include administering (e.g., topically administering or subconjunctivally injecting) a hypotonic solution that allows for the swelling of the conjunctiva in the eye to which the hypotonic solution is administered. In some embodiments, the hypotonic solution is administered one or more times (e.g., two, three, four, five, six, seven, eight, nine, or ten times) during any one or more periods of the treatment: one or more times (e.g., two, three, four, five, six, seven, eight, nine, or ten times) prior to administration of the composition containing a crosslinking agent, one or more times (e.g., two, three, four, five, six, seven, eight, nine, or ten times) intermittently during the administration of the composition containing a crosslinking agent, and one or more times (e.g., two, three, four, five, six, seven, eight, nine, or ten times) intermittently during the UVA light irradiation. In other embodiments, the hypotonic solution is administered one or more times (e.g., two, three, four, five, six, seven, eight, nine, or ten times) after the administration of the composition containing a crosslinking agent, but before the irradiation with UVA light. In some embodiments, the irradiation with UVA light occurs between 1 minutes and 2 hours (e.g., between 1 minute and 1 hour, between 1 minute and 45 minutes, between 1 minute and 30 minutes, between 1 minute and 20 minutes, and between 1 minute and 10 minutes) after the administration of the hypotonic solution or the last administration of the hypotonic solution (in instances where the hypotonic solution is administered more than once to the eye of the subject). In some embodiments of any of these methods, the composition containing the crosslinking agent and the hypotonic solution can be the same solution (e.g., a hypotonic solution containing a crosslinking agent).

These methods can be performed by any medical professional (e.g., a physician, a nurse, a technician, or a physician's assistant). These methods can be performed on a subject more than once (e.g., two, three, four, five, or six times). In examples where the methods are performed more than once, the method can be performed with a frequency of at least once every five years (e.g., once every four years, once every three years, once every two years, once every year, or once every six months). A physician can readily determine the frequency at which the methods described herein should be performed on a subject (e.g., based on the severity, frequency, and number of symptoms of a conjunctival disease observed in the subject, or the observed inflammation in a conjunctiva in an eye of the subject).

The efficacy of the present methods in a subject can be determined by observing the severity, frequency, and number of symptoms of a conjunctival disease (e.g., autoimmune disease of conjunctiva, allergic conjunctivitis, and dry eye disease) experienced by the subject. For example, successful treatment will result in a decrease in the severity, frequency, and/or number of symptoms (e.g., any of the exemplary symptoms described here or known in the art) experienced by a subject having a conjunctival disorder (e.g., autoimmune disease of conjunctiva, allergic conjunctivitis, and dry eye disease). In some of these methods, effective treatment can also be observed by a detection of an decrease in the inflammation of the conjunctiva in the treated eye and/or a decrease in the number of immune cells in the conjunctiva in the treated eye of the subject (e.g., as observed using slit lamp examination, in vivo confocal microscopy, or 2-photon microscopy), by detection of reduced ocular surface damage (e.g., as observed clinically by corneal fluorescein staining and conjunctival staining by lissamine green or rose bengal), or by detection of improved tear film (e.g., by showing increased tear secretion using Schirmer test or by showing increased tear break up time), e.g., as compared to the same measured parameter in a subject prior to treatment.

The invention is further described in the following examples, which do not limit the scope of the invention described in the claims.

### Examples

### Example 1

### Collagen Crosslinking to Increase the Rigidity of Conjunctiva

These experiments are performed to evaluate the effect of the methods described herein on normal conjunctiva.

Collagen crosslinking (using the methods described herein) is performed on the temporal bulbar conjunctiva in one eye of healthy rabbits (female New Zealand white rabbits weighing between 2000 mg to 2500 mg). The other eye in the rabbits serves as the control, and does not receive any treatment. Other control rabbits are used as the control for treatment with only UVA light irradiation in one eye, and treatment with only riboflavin in the other eye. The findings are compared with rabbits that did not receive any treatment at all.

### Anesthesia and Administration of Riboflavin

General Anesthesia

General anesthesia of rabbits is performed using Ketamine HCl/Xylazine HCl.

Application of Riboflavin

Riboflavin solution is applied in two different ways: topical application and subconjunctival injection. The following solutions are used for topical application: 0.1% riboflavin, 20% dextran, isotonic solution; 0.25% riboflavin, saline, isotonic solution; 0.1% riboflavin, saline, hydroxypropylmethylcellulose (HPMC); and 0.25% riboflavin, HPMC, benzalkonium chloride (BAC), ethylenediaminetetraacetic acid (EDTA), tris(hydroxymethyl)aminomethane (TRIS) solution. The topical solution of riboflavin is applied every 3 minutes for 15, 30, 45 or 60 minutes before UVA light irradiation and every 5 minutes during the irradiation.

Two different solutions (0.1% riboflavin in saline or 0.25% riboflavin in saline) are used for subconjunctival injection. Different volumes of these solutions (0.05, 0.1, 0.2, 0.3, 0.4, or 0.5 mL) are injected at 10, 5, 2, or 1 minute(s) before UVA light irradiation. The subconjunctival content of riboflavin is spread using a blunt spatula to achieve a uniform thickness of riboflavin across the subsection of the conjunctiva that is irradiated with UVA light.

### UVA Light Irradiation

UVA light with a wavelength of 365 nm is used. The UVA light irradiance is 0.5, 1, 2, 3, 4, 5, or 7 mW/cm². The duration of radiation is 5, 10, 20, 30, 45, or 60 minutes. A double diode laser is used to perform the UVA light irradiation. Before each treatment, the desired irradiance is controlled with a UVA meter (LaserMate-Q; LASER 2000, Wessling, Germany) at a 1-cm distance and, if necessary, regulated with a potentiometer. The cornea and limbus is shielded during UVA light irradiation.

### Subsection of Conjunctiva Treated

The rabbit's eye is rotated to expose the temporal bulbar conjunctiva with limbal traction sutures (using 6-0 silk) at 6 and 12 o'clock positions. A subsection of 10×10 mm of the conjunctiva in this location is treated.

### Evaluation of Treatment

Macroscopic

The treated subsection of the conjunctiva is evaluated macroscopically at 1 day, 3 days, 1 week, 2 weeks, 1 month, 3 months, and 6 months in terms of redness, epithelial defect(s) (using lissamine green staining), and scar formation. In addition, before the treatment, the exact location of treated subsection is marked by a skin marker. Immediately after the treatment, the treated subsection is monitored to see whether there is any change (decrease or increase) in the size of treated subsection of the conjunctiva after treatment.

In Vivo Confocal Microscopy (IVCM)

IVCM is performed by a laser scanning in vivo confocal microscope with a corneal imaging module (HRT3-RCM; Heidelberg Engineering, Heidelberg, Germany) at baseline (1 day before the procedure), and 1 week, 1 month, 3 months, and 6 months after treatment. IVCM scanning is done at 5 different locations in the treated conjunctiva (anterior, posterior, superior, inferior, and central conjunctiva) using the Sequence Mode. A sequence of 100 images is obtained at each location up to the depth of the sclera. IVCM images are used to evaluate the conjunctival epithelium, and the arrangement of collagen fibers and the density and morphology of immune cells (dendritic cells and non-dendritic cells) in the conjunctiva. The findings are compared to those of the controls.

2-Photon Microscopy

Two-photon microscopy (2-PM) with second harmonic generation (SHG) is also performed to evaluate the spatial changes in the arrangement of conjunctival collagen fibers in vivo after treatment. It is performed at baseline (1 day before treatment), and 1 week, 1 month, 3 months, and 6 months after treatment. The set-up uses a mode-locked Ti:Sapphire laser as illumination and a photomultiplier tube as a detection unit. The excitation wavelength is set at 760 nm. The instrument is built on a modified inverted microscope. The SHG signal is collected in the backward direction via the same objective. A dichroic mirror separates the excitation light from the generated nonlinear signal coming back from the sample. The XY scanning of the sample is performed using two non-resonant galvanometric mirrors. A direct current (DC) motor coupled to the objective position control allows optical sectioning across the entire cornea along the Z-direction. Two different filters (long-pass and narrow-band) are placed in the detection channel to allow for the independent acquisition of either 2-PM or SHG signals from the specimens under analysis. The findings in treated conjunctiva are compared to the controls.

Optical Coherence Tomography

Anterior segment optical coherence tomography (AS-OCT) is performed to evaluate the depth of penetration of riboflavin and also the depth of conjunctival changes after treatment. AS-OCT by Fourier-Domain RTVue-100 OCT (Optovue Inc., Fremont, Calif.) is used. Imaging is performed prior to the starting application of riboflavin, at the end of 15, 30, 45 and 60 minutes of application of riboflavin solution, or 10 minutes after subconjunctival injection of riboflavin, and 30 minutes after irradiating the conjunctiva with UVA light. The depth of the riboflavin penetration (by potential reflectivity band) and depth of conjunctival changes after treatment (by potential demarcation line) are compared among various application methods.

Biomechanics

Rabbits are killed at 1 day, 1 week, 1 month, 3 months, and 6 months after treatment and stress-strain measurements are performed for the treated conjunctiva specimens and the controls. Four-by-ten-mm strips of temporal bulbar conjunctiva are clamped horizontally between the jaws of a commercially available microcomputer-controlled biomaterial tester (Minimat 2000; Rheometric Scientific GmbH, Bensheim, Germany). The biomechanical properties of conjunctival strips are obtained from the force extension curves. The stress-strain values are fit to characterize the stiffening effect by an exponential function using the SPSS calculation program (SPSS Inc, Chicago, Ill.). Stress is calculated by dividing the force generated during extension by the initial cross-sectional area, and strain is calculated as a percent elongation of the conjunctival strip from the original length. The ultimate stress is measured at the tearing point. The ultimate strain is measured as the percentage of the starting length of the strip represented by the amount of elongation at the point of tearing. Young's modulus (E) is determined as the slope of the stress-strain graphs at 50% strain.

Light Microscopy

The conjunctival specimens from the killed rabbits at 1 hour, 1 day, 1 week, 1 month, and 3 months after treatment are placed in 10% formaldehyde for approximately 48-72 hours. After fixation, each conjunctival specimen is sectioned on the sagittal plane and stained with hematoxylin/eosin (as a general histological tissue stain) and Masson's trichrome (to stain the corneal collagen). Infiltration of inflammatory cells and collagen content are compared between the treated conjunctiva and the controls. In addition, specimens from the sclera, ciliary body, choroid, and retina are stained to evaluate any possible side effects on these structures.

TUNEL Assay for Apoptosis

Specimens of conjunctiva and sclera are evaluated at 4 hours, 24 hours, 1 week, 1 month, 3 months, and 6 months using a terminal deoxynucleotidyl transferase (TdT)-mediated dUTP-biotin nick end labeling (TUNEL) assay to determine apoptosis in the conjunctival epithelium, conjunctival fibroblasts, and scleral fibroblasts. A TUNEL assay is able to detect apoptosis with DNA strand breaks in situ. In each TUNEL assay, after the quenching of endogenous peroxidase, sections are incubated with TdT buffer (30 mM Tris, 140 mmol/L sodium cacodylate, and 1 mmol/L cobalt chloride) at pH 7.2, and incubated with 0.3 μg/mL TdT and biotinylated-dUTP (1:200) in TdT buffer for 60 minutes at 37° C. Labeled nuclei are detected with Vectastain ABC, and peroxidase activity is visualized by 3-amino-9-ethylcarbazole which yields a reddish brown reaction product. The sections are lightly counterstained with hematoxylin. As a positive control, tissue sections of follicular hyperplasia of the appendix are used and give the expected positive staining of tingible bodies in the germinal centers.

Transmission Electron Microscopy

Transmission electron microscopy (TEM) is performed at 1 day, 1 week, 1 month, 3 months, and 6 months to examine the conjunctival strips in terms of diameter of collagen fibers and apoptotic changes like formation of apoptotic bodies, chromatin condensation, and cell shrinkage.

Conjunctival specimens are fixed in 2.5% glutaraldehyde in 0.1 M PBS buffer at 4° C. Then, they are dissected at 50 μm and 100 μm of depth and embedded in epon. Ultrathin epon sections 50-70 nm thick are cut and contrasted with uranyl acetate and lead citrate, and evaluated morphometrically using the TEM. The TEM images are transferred to a computer screen. The diameters are marked manually with a computer mouse and calculated with the help of the semiautomatic software program Analysis (Soft Imaging System GmbH, Munster, Germany). In each case, the diameters of 80 to 160 contiguous fiber section profiles are measured. Only fiber profiles with clearly defined borders of high contrast are included; profiles with low contrast and indistinct borders are discarded. In some sections with a slightly ellipsoidal section profile (as a result of oblique sectioning), the minimal transverse diameter of the collagen fibers is measured because in ellipsoidal section profiles, the shortest diameter is equal to the diameter of the corresponding circular section profile.

Enzymatic Digestion

Conjunctival specimens of 5×5 mm are obtained from the treated and the control eyes at 1 day after treatment. They are placed in the following solutions until they are completely digested.

Pepsin Assay: The pepsin solution is made up of 1 g purified pepsin in 10 mL of 0.1M HCl at pH 1.5. A pH of about 1.5 is the pH-optimum for pepsin activity.

Trypsin Assay: The conjunctival specimens are placed into plastic containers with a solution made up of 1 g trypsin in 10 mL of PBS-buffer at pH 7.5. A pH of about 7.5 is the pH-optimum for trypsin activity. As trypsin digestion does not occur in non-denatured conjunctiva, the crosslinked and control conjunctivas are exposed to trypsin after prior heat denaturation in boiling water at 100° C. for 10 minutes.

Collagenase Assay: The crosslinked and the control specimens are placed in a 0.1% bacterial collagenase A solution (100 U/g or 0.1 U/mL) in PBS at pH 7.5.

Changes of Conjunctival Specimens: After crosslinking treatment and exposure to the enzyme solutions, the diameter of each specimen is monitored and photographed daily. The statistical analysis of the resorption times of the specimen diameter from the starting diameter to 50% reduction and from 50% reduction to 10% reduction are compared between the groups. In addition, the time for complete digestion of the specimens are also compared between the groups.

Contraction Model

Each conjunctiva is dissected and cut into 5- to 10-mm² pieces and placed in a 12-well plate. Polyester membrane inserts (3-μm pore size and 12-mm membrane diameter) are placed on top of the segments, holding the tissue in place and maintaining their shape. The segments are submerged in 1.5 mL of DMEM and are kept at 37° C. and 5% carbon dioxide for 4 weeks. Digital photographs are obtained, and the media are changed twice a week. Changes in area are calculated for each segment. The percentage contraction is calculated by normalizing percentage area values to those at day 0. The sizes of the conjunctival segments are compared between groups with and without treatment.

Immunohistochemical Studies

Monoclonal anti-Ki-67, anti-CD-34, anti-collagen type I, anti-alpha smooth muscle actin, and polyclonal anti-desmin (for myofibroblasts) are used to stain sections of the conjunctiva from the treated or untreated rabbits.

### Example 2

### Collagen Crosslinking to Decrease the Proliferation or Number of Human Conjunctiva/Tenon Fibroblasts

Experiments are performed to test the effects of the present methods on cultured human conjunctiva/tenon fibroblasts. In these experiments, cultures of human conjunctiva/tenon fibroblasts are generated from subconjunctival Tenon biopsy specimens obtained from glaucoma patients during surgery. The fibroblasts are cultured in RPMI medium with L-glutamine supplemented with 10% heat-inactivated newborn calf serum and 50,000 U/L penicillin-streptomycin. The cells are exposed to four different treatments:
1) No Treatment
2) Riboflavin Treatment Alone: The cells are exposed to 25, 50, 100, or 500 μM of riboflavin.
3) Riboflavin Treatment plus UVA Light Irradiation: The cells are exposed to 25, 50, 100, or 500 μM riboflavin solution plus UVA light irradiances (365 nm) of 0.1, 0.3, 0.5, 0.7, 1, 3, 5, or 7 mW/cm² for 5, 10, 20, 30, 45, or 60 minutes.
4) UVA Light Irradiation Alone: The cells are exposed to UVA irradiances (365 nm) of 0.1, 0.3, 0.5, 0.7, 1, 3, 5, or 7 mW/cm² for 5, 10, 20, 30, 45, or 60 minutes.

For quantitative analysis of cell death, Annexin V-FITC/propidium iodide fluorescence-assisted cell sorting (FACS) analysis, lactate dehydrogenase release assays, and Western immunoblotting are performed at 24 hours after treatment. The rates of cell death are compared among the four different treatment regimens.

### Experiment 3

### Collagen Crosslinking to Decrease the Proliferation or Number of Human Pterygium Fibroblasts

Pterygium is a fibrovascular growth from the conjunctiva onto the cornea. Fibroblasts play a vital role in the formation of pterygium and its recurrence after surgical excision. Experiments are performed to test the effects of the present methods on cultured human pterygium fibroblasts.

In these experiments, pterygium specimens are obtained after the surgical removal of primary pterygium. The central portion of the pterygium body is used for all cultures. These tissue samples are used for explant cultures to generate pterygium body fibroblasts. Each specimen is cut into explants of approximately 1×1-mm and placed into 100-mm tissue culture dishes. Ten minutes later, each explant is covered with a drop of FBS and placed overnight in an incubator at 37° C. under 95% humidity with 5% CO₂. Ten milliliters of medium (D-FBS) containing DMEM enriched with 10% FBS, 50 mg/ml gentamicin, and 1.25 mg/mL amphotericin B is added the next day, and the medium is changed every 2 days thereafter. The fibroblasts are sub-cultured with 0.05% trypsin and 0.85 mM EDTA in a calcium-free MEM medium at 80% to 90% confluence with 1:3 to 1:4 split for three passages. The cells are exposed to four different treatments:
1) No Treatment
2) Riboflavin Treatment Alone: The cells are exposed to 25, 50, 100, or 500 μM of riboflavin.
2) Riboflavin Treatment plus UVA Light Irradiation: The cells are exposed to 25, 50, 100, or 500 μM riboflavin solution plus UVA light irradiances (365 nm) of 0.1, 0.3, 0.5, 0.7, 1, 3, 5, or 7 mW/cm² for 5, 10, 20, 30, 45, or 60 minutes.
3) UVA Light Irradiation Alone: The cells are exposed to UVA light irradiances (365 nm) of 0.1, 0.3, 0.5, 0.7, 1, 3, 5, or 7 mW/cm² for 5, 10, 20, 30, 45, or 60 minutes.

The riboflavin solution for the UVA-irradiated samples is added to the wells 5 minutes before the irradiation, and is replaced by the cell medium after the irradiation. To avoid UVA-absorption by riboflavin solution overlying the monolayer of fibroblasts attached to the floor of the wells, the wells are irradiated from underneath, by fixing the UVA-double diode (365 nm) 1 cm under the respective wells with the help of a stand.

The cell cultures are evaluated for cell death 24-h after treatment using trypan blue and Yopro-fluorescence staining to determine possible cell damage after treatment. One-hundred mL of 0.25% trypan-blue solution dissolved in colorless culture medium is applied per well for 15 minutes, followed by two-fold rinsing with culture medium. After microscopic evaluation of the trypan-blue staining, the cells are subsequently stained with Yopro by adding 1 mL per well, followed by one rinse with culture medium. For trypan blue, the cultures are examined in an inverse microscope at 100- to 400-fold magnification using differential interference contrast, and for Yopro, using fluorescence at 488 nm (N2.1 filter). Only the nuclei of damaged cells are labeled with the stains. The rates of cell damage are compared among the four different treatment regimens. In addition, TUNEL assay and Ki-67 positive cell counting are also performed to identify apoptotic cells.

### Experiment 4

### Conjunctival Collagen Crosslinking to Treat Conjunctival Scarring

Conjunctival scarring may develop following a variety of conjunctival insults including severe conjunctival inflammation after a chemical burn. This scar formation may result in symblepharon and fornix shortening. Fibroblasts play an important role in this cicatricial process. Experiments are performed to test the effects of the present methods on a rabbit model of conjunctival scarring.

In these experiments, in both eyes of rabbits, alkaline burn is induced in inferior bulbar and forniceal conjunctiva by application of sodium hydroxide (2.5 N) for 3 minutes. The eyes are then irrigated by saline solution. One eye serves as the control. The other eye receives the collagen crosslinking treatment (e.g., as described above) at 3 days or 7 days after inducing the chemical burn. Development of conjunctival scar and symblepharon is evaluated at 30 days by macroscopic examination and by histopathologic evaluation to determine collagen formation and viability of conjunctival fibroblasts.

### Experiment 5

### Conjunctival Collagen Crosslinking to Treat Allergic Conjunctivitis

It has been shown that phototherapy with UVA light has immunosuppressive effects, and thus it has been used to treat various allergic and immunologic disease of skin (such as atopic dermatitis, graft-versus-host disease, and psoriasis), oral mucosa (such as lichen planus and graft-versus-host disease), or nasal mucosa (such as allergic rhinitis). Experiments are performed to test the effects of the present methods on an allergic conjunctivitis rabbit model.

In these experiments, experimental allergic conjunctivitis is provoked in rabbits by mast cell activators. Because the inflammatory response associated with allergy involves several mechanisms, three kinds of reagents are used to provoke an allergic response. The rabbits are divided into three groups. Group I is provoked by topical application of codeine phosphate (2.5 mg/mL). Group II is provoked by compound 48/80 (50 mg/mL): the best-known polybasic histamine releaser, which is a mixture of polymers synthesized by condensing N-methyl-p-methoxyphenylamine with formaldehyde. Group III is provoked by lipopolysaccharide (10 ng/eye). Signs of allergic conjunctivitis, such as papillary reaction, conjunctival chemosis, and injection are observed within 30 minutes, and the maximal response is observed 1.5 hours after provocation. Without treatment, all symptoms and signs disappear within 24 hours.

Both eyes of each rabbit are used for the study: one eye receives crosslinking treatment (at 2 hours after provocation) and the other eye does not receive crosslinking treatment. The crosslinking procedure is performed as described above. Rabbits are evaluated before provocation, 30 minutes, and 1, 3, 6, 9, 12, 18, 24 and 48 hours after provocation for clinical signs of the allergy. In addition, tear levels of cytokine IL-10 at 2, 6, and 12 hours after provocation, as well as tissue levels of eosinophils in the conjunctiva at 12 hours, are compared between the treated eyes and the controls.

In additional experiments to test the effects of the presently described methods on delayed hypersensitivity, rabbits are sensitized by subcutaneous injections of complete Freund's adjuvant (6 mL/head). Two weeks later, a delayed-type response is elicited by injection of purified protein derivative (10 mg/mL, 30 mL each) into the upper bulbar conjunctiva of both eyes. The clinical features of bulbar conjunctival edema and hyperemia appear at 20 hours after challenge. At 24-hours after challenge, the bulbar conjunctival edema and hyperemia in both eyes are scored. Immediately after scoring, conjunctival crosslinking is performed on one eye and the other eye will serve as the control. The examination is repeated at 1, 3, 6, and 12 hours to compare the allergic signs in the conjunctiva between the two groups. In addition, conjunctival specimens are obtained at 12 hours to evaluate the levels of inflammatory cells, including T cells, eosinophils, and Langerhans cells.

### Experiment 6

### Conjunctival Collagen Crosslinking to Treat Dry Eye

Experiments are performed to test the effects of the presently described methods on a dry eye rabbit model. In these experiments, dry eye is induced in both eyes of rabbits by administering atropine sulfate (1.0%) ophthalmic solution into the lower conjunctival sac three times a day. After 1 week, the crosslinking procedure (e.g., as described above) is performed on one eye and the other eye serves as the control. Schirmer's test, tear break-up test (TBUT), corneal fluorescein staining, and conjunctival impression cytology (for goblet cell density) at baseline, 3 days, 1 week, and 2 weeks after the crosslinking treatment are compared between the treated eyes and the controls.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a conjunctival disease in a subject, the method comprising:
topically administering or subconjunctivally injecting a composition containing a crosslinking agent into an eye of a subject having or suspected of having a conjunctival disease; and
irradiating a subsection of the conjunctiva of the eye of the subject with ultraviolet A (UVA) light for a time and duration sufficient to treat the conjunctival disease in the eye of the subject.

2. The method of claim 1, wherein the subject has or is suspected of having decreased rigidity in conjunctiva of the eye of the subject, and the method includes irradiating a subsection of the conjunctiva of the eye of the subject with ultraviolet A (UVA) light for a time and duration sufficient to increase the rigidity of the conjunctiva in the eye of the subject.

3. The method of claim 1, wherein the subject has or is suspected of having increased proliferation or number of conjunctival fibroblasts in conjunctiva of the eye of the subject, and the method includes irradiating a subsection of the conjunctiva of the eye of the subject with ultraviolet A (UVA) light for a time and duration sufficient to decrease the proliferation or number of conjunctival fibroblasts in the subsection of the conjunctiva of the eye of the subject.

4. The method of claim 1, wherein the subject has or is suspected of having an increased number of immune cells in conjunctiva of the eye of the subject, and the method includes irradiating a subsection of the conjunctiva of the eye of the subject with ultraviolet A (UVA) light for a time and duration sufficient to decrease the number of immune cells in the subsection of the conjunctiva of the eye of the subject.

5. The method of claim 1, wherein the irradiating is performed about 1 second to about 2 hours after the administering.

6. The method of claim 1, wherein the administering is performed intermittently during, or both before and intermittently during, the irradiating period.

7. (canceled)

8. The method of claim 1, wherein the crosslinking agent is riboflavin, rose bengal, octadiazopyruvoyl PAMAM (8G.1 DAP, 1.3), or a 1,8-naphthalimide dye.

9. The method of claim 1, wherein the irradiating is performed using a laser that emits UVA light, optionally a double diode laser.

10. (canceled)

11. The method of claim 1, wherein the UVA light only irradiates the conjunctiva to a maximum depth of 250 μm from the surface of the conjunctiva.

12. The method of claim 11, wherein the UVA light only irradiates the conjunctiva to a maximum depth of 200 μm from the surface of the conjunctiva.

13. The method of claim 1, wherein the UVA light does not irradiate the sclera of the eye of the subject.

14. The method of claim 13, further comprising positioning a mask or filter over a portion of the eye in order to prevent the UVA light from irradiating the sclera, cornea, and/or limbus.

15. The method of claim 1, wherein the subsection of the conjunctiva is irradiated for less than about 1 hour, optionally for less than 10 minutes.

16. (canceled)

17. The method of claim 1, wherein the irradiance of the UVA light is between about 0.1 mW/cm² and about 45 mW/cm², optionally between about 0.1 mW/cm² and about 15 mW/cm².

18. (canceled)

19. The method of claim 1, wherein the UVA light is pulsed UVA light.

20. The method of claim 1, wherein the irradiating provides a dose of between about 0.1 J/cm² to about 15 J/cm², optionally between about 0.1 J/cm² and about 8 J/cm².

21. (canceled)

22. The method of claim 1, wherein the UVA light has a wavelength of about 340 nm to about 380 nm.

23. The method of claim 1, wherein the subsection of the conjunctiva has an area of between about 3 mm2 to about 300 mm2.

24. The method of claim 1, wherein the conjunctival disease is selected from the group consisting of: conjunctivochalasis, conjunctival bleb thinning after a glaucoma surgical procedure, pterygium, a cicatricial conjunctival disorder, autoimmune disease of conjunctiva, allergic conjunctivitis, and dry eye disease.

25. The method of claim 2, wherein the subject has or is suspected of having conjunctivochalasis or conjunctival bleb thinning after a glaucoma surgical procedure.

26. The method of claim 3, wherein the subject has or is suspected of having pterygium or a cicatricial conjunctival disorder.

27. The method of claim 24, wherein the cicatricial conjunctival disorder is selected from the group consisting of: Stevens-Johnson syndrome, ocular cicatricial pemphigoid, and thermal or chemical burn of the conjunctiva.

28. The method of claim 4, wherein the subject has or is suspected of having an autoimmune disease of conjunctiva, allergic conjunctivitis, and dry eye disease.

29.-31. (canceled)

32. The method of claim 1, wherein the composition containing a crosslinking agent is a hypotonic solution.

33. The method of claim 1, further comprising topically administering or subconjunctivally injecting to the eye of the subject a hypotonic solution prior to, during, or both prior to and intermittently during irradiating the subsection of the conjunctiva in the eye of the subject.

34.-35. (canceled)
